(19) United States
(12) Patent Application Publication
(10) Pub. No.: US 2023/0000917 A1
(43) Pub. Date: Jan. 5, 2023

(54) TREATMENT OF HEPATITIS B VIRUS (HBV) INFECTION

(71) Applicant: UCL BUSINESS LTD, London (GB)

(72) Inventors: Mala Maini, London (GB); Nathalie Schmidt, London (GB); Alice Burton, London (GB); Jane Mckeating, Oxford (GB); Peter Wing, Oxford (GB)

(21) Appl. No.: 17/780,786

(22) PCT Filed: Nov. 27, 2020
(86) PCT No.: PCT/GB2020/053034

§ 371 Date: May 27, 2022

(30) Foreign Application Priority Data
Nov. 29, 2019 (GB) 1917498.6

(51) Int. Cl. A61K 35/17; A61K 45/06; A61P 31/20

(52) U.S. Cl. CPC A61K 35/17; A61K 45/06; A61P 31/20

(57) ABSTRACT

The present invention provides an acyl-CoA: cholesterol acyltransferase (ACAT) inhibitor for use in the treatment of hepatitis B vims (HBV) infection in a subject.

### FIELD OF THE INVENTION

The present invention relates to an acyl-CoA: cholesterol acyltransferase (ACAT) inhibitor for use in the treatment of hepatitis B virus (HBV) infection in a subject.

### BACKGROUND TO THE INVENTION

Hepatitis B virus infects an estimated 260 million people worldwide, causing around 780,000 deaths a year. A complication of chronic HBV (CHB) infection is the development of HBV-associated (also termed HBV-related) hepatocellular carcinoma (HCC). HCC is the second leading cause of cancer related death with a rising incidence and an ongoing high mortality, with less than 20% of patients responding to current immunotherapies. There is thus a pressing need for novel therapies or novel therapy combinations for the treatment of HBV infection.

Existing treatments very rarely achieve sustained off-treatment viral suppression because they cannot eliminate the cccDNA transcriptional template (episomal form of HBV) or recover the profound immune exhaustion associated with HBV infection. Because of the inadequacy of currently available treatment for HBV, many diagnosed patients are monitored without treatment for years, creating an ongoing pool of highly infectious, stigmatised individuals.

A primary goal of the current drug development in the field is to induce a state of “functional cure” of HBV where residual viral replicative intermediates within infected hepatocytes are kept under tight long-term immune control, as seen in adults resolving the infection spontaneously. Functional cure would ideally be characterised by sustained off-treatment viral suppression, HBV surface antigen (HBsAg) loss—with or without anti-HBs antibody seroconversion—and more effective reduction in the risk of HCC.

The current best available treatments of CHB are nucleoside/nucleotide analogues (NAs or NUCs), such as Tenofovir or Entecavir, that suppress viraemia effectively with a high barrier to resistance, and are well-tolerated. Other less effective NAs such as Lamivudine, Telbivudine and Adefovir (with much higher risks of resistance development) are still used in some countries, for cost considerations. However even the latest generation of NAs (Tenofovir, Tenofovir alafenamine or Entecavir) rarely induce sustained off-treatment responses as they cannot target the episomal form of HBV (cccDNA) to reduce HBsAg, do not have immunomodulatory capacity and can only partially reduce, but not prevent, the risk of subsequent HCC. Treatment with NAs is currently typically therefore lifelong, with resultant issues of compliance, cost, stigma of ongoing transmission risk, potential emergence of resistance and/or toxicity.

An alternative or additional therapy is pegylated interferon-alpha, given as a finite (typically 1 year) course, which can lead to a durable off-treatment response with HBsAg loss in approximately 10% of patients. Its capacity to induce cure in a proportion of patients has been attributed to its combined direct antiviral and immunomodulatory potential. However, it is rarely used due to poor tolerability and limited, genotype-dependent, response rates.

There is thus a need for alternative strategies for therapies or combination therapies to treat HBV and HBV-associated HCC. In particular, there is a need for therapies which enable sustained off-treatment viral suppression.

### SUMMARY OF THE INVENTION

The present inventors have surprisingly found that acyl-CoA: cholesterol acyltransferase (ACAT) inhibition is efficacious in the treatment of HBV infection. Unexpectedly, ACAT inhibition has the potential to target both the hepatitis B virus itself and the host immune response against it. Therefore, ACAT inhibition has the potential to target HBV in multiple ways and thus the potential to enable sustained off-treatment viral suppression.

Accordingly, in a first aspect, the present invention provides an ACAT inhibitor for use in the treatment of hepatitis B virus (HBV) infection in a subject.

The present invention further provides a method of treating HBV infection in a subject which comprises administering a therapeutically effective amount of an ACAT inhibitor to the subject.

The invention also relates to the use of an ACAT inhibitor in the preparation of a medicament for treating HBV infection in a subject.

In a further aspect, the present invention provides an ACAT inhibitor for use in the treatment or prevention of HBV infection in a subject, wherein the infection is caused by HBV genotype C.

The present invention further provides a method of treating HBV infection in a subject which comprises administering a therapeutically effective amount of an ACAT inhibitor to the subject, wherein the infection is caused by HBV genotype C.

The invention also relates to the use of an ACAT inhibitor in the preparation of a medicament for treating HBV infection in a subject, wherein the infection is caused by HBV genotype C.

The ACAT inhibitor may be administered to the subject in combination with at least one further pharmaceutically active agent.

In another aspect, the present invention provides a therapeutic vaccine composition comprising an ACAT inhibitor for use in the treatment or prevention of HBV infection in a subject.

The present invention further provides a method of treating a HBV infection in a subject which comprises administering a therapeutically effective amount of a therapeutic vaccine composition comprising an ACAT inhibitor to the subject.

The invention also relates to the use of an ACAT inhibitor in the preparation of a therapeutic vaccine composition for treating HBV infection in a subject.

In a further aspect, the present invention provides a therapeutic HBV vaccine composition comprising an ACAT inhibitor.

In a further aspect, the present invention provides a TCR-gene-engineered cell or CAR cell having reduced ACAT activity and/or reduced ACAT expression for use in the treatment of HBV infection in a subject.

The present invention further provides a method of treating HBV infection in a subject which comprises administering a therapeutically effective amount of TCR-gene-engineered cell or CAR cell having reduced ACAT activity and/or reduced ACAT expression to the subject.

The subject to be treated in the present invention may have a chronic HBV infection (CHB) and/or be at risk of developing HBV-associated hepatocellular carcinoma (HCC), have HBV-associated HCC or have previously had HBV-associated HCC.

### BRIEF DESCRIPTION OF THE FIGURES

FIG. 1: A) Schematic representation of the cell treatment. PBMC from patients with CHB were stimulated with HBV peptides and cultured for 7 days in vitro in the presence or absence of the ACAT inhibitor Avasimibe. Detection of IFNγ and TNF production via flow cytometry as a readout of antiviral function of T cells and detection of CD107a mobilization to the cell surfaces as a marker of cytotoxicity. B) Example plot. Detection of IFNγ production by CD8+ T cells 7 days after culture with HBV peptide or HBV peptide and Avasimibe. C)

Summary data of IFNγ production by CD8+ T cells in the presence or absence of Avasimibe based on patients with detectable pre-existing HBV-specific CD8+ T cell responses. D) Fold change of IFNγ production by CD8+ T cells after stimulation with HBV peptides ±ACAT inhibition (ACAT inh) compared to unstimulated control. E) Fold change of TNF production by CD8+ T cells after stimulation with HBV peptides ±ACAT inhibition compared to unstimulated control. F) Fold change of CD107a mobilization after stimulation with HBV peptides ±ACAT inhibition compared to unstimulated control as readout of cytotoxicity. G)

Summary data of IFNγ production by CD4+ T cells in the presence or absence of Avasimibe based on patients with detectable pre-existing HBV-specific CD4+ T cell responses. H) Summary data of IFNγ production by CD4+ T cells in the presence or absence of Avasimibe on all patients.

FIG. 2: A) Intrahepatic lymphocytes (IHL) from patients with chronic HBV were stimulated with HBV peptides in vitro for 16 h±the ACAT inhibitor K-604. Detection of IFNγ production of CD8+ T cells as a readout of antiviral function. B) Tumour-infiltrating lymphocytes (TIL) from patients with HCC were stimulated with tumour peptides in vitro for 16 h±the ACAT inhibitor K-604. Detection of IFNγ production of CD8+ T cells as a readout of antitumour function. C) IHL from patients with chronic HBV were stimulated with HBV peptides in vitro for 16 h±the ACAT inhibitor K-604. Detection of IFNγ production of CD4+ T cells as a readout of antiviral function. D) Tumour-infiltrating lymphocytes (TIL) from patients with HCC were stimulated with tumour peptides in vitro for 16 h±the ACAT inhibitor K-604. Detection of IFNγ production of CD4+ T cells as a readout of antitumour function.

FIG. 3: A) Ex vivo CTB staining of PBMC from patients with CHB to identify GM-1 enriched microdomains (lipid rafts) in PD-1 positive and PD-1 negative CD8+ T cells. B) PBMC from patients with CHB stimulated with HBc OLP alone (light grey) or in the presence of ACAT inhibition (ACAT inh, dark grey), PD-L1/PD-L2 blockade (PD-1 block, white) or a combination of ACAT inhibition and PD-L1/PD-L2 blockade (combo, black). Summary data of patients showing IFNγ production by CD8+ T cells in any of the four conditions (n=26).

Boxes below the histogram indicate treatment strategy resulting in the highest IFNγ production in the respective patients (light grey: peptide alone; white: PD-1 block; dark grey: ACAT inhibition; black: combo).

FIG. 4: Treatment of PBMC from patients with CHB with DMSO or the ACAT inhibitor Avasimibe (ACAT inh) for 7d. A) Frequency of T_{FH} cells (CD3+CD4+CXCR5+PD-1+) B)

Expression of costimulatory and activation markers (ICOS, OX40, 4-1BB, CD4OL) on T_{FH} cells in the presence or absence of Avasimibe.

FIG. 5: A) Representative plot of calcium mobilisation following BCR-crosslinking in magnetically-purified B cells isolated from a patient with CHB. Values represent the mean fluorescence intensity of responding cells normalised to unstimulated cells. B) Summary data comparing calcium mobilisation in BCR-stimulated cells from healthy controls (HC; n=15) or patients with CHB (CHB; n=15) without Avasimibe. Dotted lines represent±1 S.D. of the mean of HC; a threshold level was set at −1 S.D of the mean of HC to identify patients with CHB with low baseline responses (shown in black). C,D) Summary data showing calcium mobilisation with or without avasimibe in classical memory B cells (CD27+CD21+) from: C) patients with CHB (n=15) or D) healthy controls (n=15). E) Cross-sectional analysis of calcium mobilization with and without Avasimibe in patients with CHB where baseline responses are below 1 S.D of the mean of HC responses (n=9). F) Staining of neutral lipid droplets (including cholesteryl ester) with LipidTox after treatment with ACAT inhibitor K604 for 1 h.

FIG. 6: A) Quantification of IFNγ production by TCR-gene engineered CD8+ T cells specific for the HLA-A2-restricted HBV-core18-27 epitope following stimulation with increasing doses of core18-27 peptide (0.0001ng/ml to 0.05ng/ml) presented by a HepG2 cell line (E:T ratio 1:1) for 18 h in cRPMI+10% FCS in the presence of 1 μg/m1 Brefeldin A, 100 U/ml IL-2, 10 ng/ml IL-7 and 10 ng/ml IL-15. 1 μM ACAT inhibitor Avasimibe was added where indicated. B) Quantification of specific target cell lysis by ToxiLight Cytotoxicity Assay.

FIG. 7: A) qPCR quantification of extracellular HBV DNA in the supernatant following transduction of HepG2-NTCP cells with Ad-HBV (MOI 20) and incubation with the indicated doses of Avasimibe or 1 μM Entecavir, which were replenished every 3 days. Drugs were applied for a total of 6 days at the start of infection. B+C) de-novo infection of HepG2-NTCP cells with HBV (MOI 200) and incubation with the indicated doses of Avasimibe or 1 μM Entecavir, which were replenished every 3 days. Drugs were applied for a total of 6 days at the start of infection. B) Schematic representation of the experiments. C) qPCR quantification of extracellular HBV DNA.

FIG. 8: A) Quantification by ELISA of secreted HBsAg following de-novo infection of HepG2-NTCP cells with HBV and incubation with the indicated doses of Avasimibe or 1 μM Entecavir, which were replenished every 3 days. Drugs were applied for a total of 6 days from the start of infection. B) Schematic representation of the experiments for determining the effects of ACAT inhibition when treatment is initiated 6 days after the infection is established. C) qPCR quantification of extracellular HBV DNA following de-novo infection of HepG2-NTCP cells with HBV. Infection was established for 6 days prior to treatment with either the indicated doses of Avasimibe or 1 μM Entecavir. Drugs were replenished every 3 days with treatment lasting for a total of 6 days. D) Quantification of secreted HBsAg by ELISA following de-novo infection of HepG2-NTCP cells with HBV. Infection was maintained for 6 days before the cells were treated with indicated doses of Avasimibe or 1 μM Entecavir. Drugs were replenished every 3 days with treatment lasting for a total of 6 days.

FIG. 9: A) qPCR quantification of cccDNA following co-culture of HBV infected HepG2-NTCP cells and TCR-transduced T cells. Prior to the start of the co-culture, HepG2-NTCP were infected and treated with Avasimibe for 11 days and TCR- gene engineered T cells were treated with Avasimibe for 24 h. T cells and infected HepG2-NTCP cells were co-cultured for 72 h at an E:T ratio of 1:1 in the presence of Avasimibe where indicated. B) qPCR quantification of intracellular DNA following co-culture of HBV infected HepG2-NTCP cells and TCR-gene engineered T cells. Prior to the start of the co-culture, HepG2-NTCP were infected and treated with Avasimibe for 11 days and TCR-gene engineered T cells were treated with Avasimibe for 24 h. T cells and infected HepG2 cells were co-cultured for 72 h at an E:T ratio of 1:1 in the presence of Avasimibe where indicated.

### DETAILED DESCRIPTION OF THE INVENTION

### ACYL-CoA: Cholesterol Acyltransferase (ACAT) Inhibitor

The present invention provides an ACAT inhibitor for use in the treatment of hepatitis B virus (HBV) infection in a subject.

A summary of some of potential beneficial effects which may be provided by ACAT inhibition in the treatment of HBV infection include, but are not limited to:
inhibiting assembly/release of virions and sub-viral particles to reduce HBV load and HBsAg levels;
enhancing both CD4 and CD8 T cell immunity (which may be specific for HBV and/or cancer antigens)
enhancing B cell immunity;
reducing carcinogenesis;
synergising with genetically engineered T cells;
synergising with anti-PD1 treatment; and
synergising with therapeutic vaccines and other immunotherapeutic or antiviral approaches.

Cholesterol is an essential component for the cell membrane and for signalling molecule synthesis. Cells obtain cholesterol through uptake of extracellular cholesterol stored in lipoproteins and de-novo intracellular biosynthesis. In the cytoplasm, free cholesterol is converted into cholesteryl ester by the enzymes acyl-CoA: cholesterol acyltransferases (ACATs, also referred to as sterol O-acyltransferase, SOAT) and stored in lipid droplets which can be toxic to immune cells. ACAT1 is ubiquitously expressed whereas ACAT2 is mainly expressed in the liver and small intestine.

ACAT1 and ACAT2 may also be referred to as sterol-O-acyltransferase (SOAT)1 and SOAT2, respectively.

The ACAT inhibitor for use in the present invention may inhibit ACAT1 and/or ACAT2.

An illustrative human ACAT1 amino acid sequence is provided by Uniprot Accession Number P24752 as shown as SEQ ID NO: 1.

SEQ ID NO: 1
MAVLAALLRSGARSRSPLLRRLVQEIRYVERSYVSKPTLKEVVIVSATR
TPIGSFLGSLSLLPATKLGSIAIQGAIEKAGIPKEEVKEAYMGNVLQGG
EGQAPTRQAVLGAGLPISTPCTTINKVCASGMKAIMMASQSLMCGHQDV
MVAGGMESMSNVPYVMNRGSTPYGGVKLEDLIVKDGLTDVYNKIHMGSC
AENTAKKLNIARNEQDAYAINSYTRSKAAWEAGKFGNEVIPVTVTVKGQ
PDVVVKEDEEYKRVDFSKVPKLKTVFQKENGTVTAANASTLNDGAAALV
LMTADAAKRLNVTPLARIVAFADAAVEPIDFPIAPVYAASMVLKDVGLK
KEDIAMWEVNEAFSLVVLANIKMLEIDPQKVNINGGAVSLGHPIGMSGA
RIVGHLTHALKQGEYGLASICNGGGGASAMLIQKL

Amino acids 1-33 of SEQ ID NO: 1 are a mitochondrial transit peptide. The ACAT1 polypeptide may comprise or consist of amino acids shown as positions 34-427 of SEQ ID NO: 1.

An illustrative human ACAT1 gene sequence is provided by NCBI Genbank Accession Number NC_000011.10. An illustrative human ACAT1 nucleic acid sequence is provided by

NCBI Genbank Accession Number NM_000019.4 and shown as SEQ ID NO: 3.

SEQ ID NO: 3
agtctacgcctgtggagccgatactcagccctctgcgaccatggctgtg
ctggcggcacttctgcgcagcggcgcccgcagccgcagccccctgctcc
ggaggctggtgcaggaaataagatatgtggaacggagttatgtatcaaa
acccactttgaaggaagtggtcatagtaagtgctacaagaacacccatt
ggatcttttttaggcagcctttccttgctgccagccactaagcttggtt
ccattgcaattcagggagccattgaaaaggcagggattccaaaagaaga
agtgaaagaagcatacatgggtaatgttctacaaggaggtgaaggacaa
gctcctacaaggcaggcagtattgggtgcaggcttacctatttctactc
catgtaccaccataaacaaagtttgtgcttcaggaatgaaagccatcat
gatggcctctcaaagtcttatgtgtggacatcaggatgtgatggtggca
ggtgggatggagagcatgtccaatgttccatatgtaatgaacagaggat
caacaccatatggtggggtaaagcttgaagatttgattgtaaaagacgg
gctaactgatgtctacaataaaattcatatgggcagctgtgctgagaat
acagcaaagaagctgaatattgcacgaaatgaacaggacgcttatgcta
ttaattcttataccagaagtaaagcagcatgggaagctgggaaatttgg
aaatgaagttattcctgtcacagttacagtaaaaggtcaaccagatgta
gtggtgaaagaagatgaagaatataaacgtgttgattttagcaaagttc
caaagctgaagacagttttccagaaagaaaatggcacagtaacagctgc
caatgccagtacactgaatgatggagcagctgctctggttctcatgacg
gcagatgcagcgaagaggctcaatgttacaccactggcaagaatagtag
catttgctgacgctgctgtagaacctattgattttccaattgctcctgt
atatgctgcatctatggttcttaaagatgtgggattgaaaaaagaagat
attgcaatgtgggaagtaaatgaagcctttagtctggttgtactagcaa
acattaaaatgttggagattgatccccaaaaagtgaatatcaatggagg
agctgtttctctgggacatccaattgggatgtctggagccaggattgtt
ggtcatttgactcatgccttgaagcaaggagaatacggtcttgccagta
tttgcaatggaggaggaggtgcttctgccatgctaattcagaagctgta
gacaacctctgctatttaaggagacaaccctatgtgaccagaaggcctg
ctgtaatcagtgtgactactgtgggtcagcttatattcagataagctgt
ttcattttttattattttctatgttaacttttaaaaatcaaaatgatga
aatcccaaaacattttgaaattaaaaataaatttcttcttctgcttttt
tcttggtaaccttgaaaa

An illustrative human ACAT2 amino acid sequence is provided by Uniprot Accession Number Q9BWD1as shown as SEQ ID NO: 2.

SEQ ID NO: 2
MNAGSDPVVIVSAARTIIGSFNGALAAVPVQDLGSTVIKEVLKRATVAP
EDVSEVIFGHVLAAGCGQNPVRQASVGAGIPYSVPAWSCQMICGSGLKA
VCLAVQSIGIGDSSIVVAGGMENMSKAPHLAYLRTGVKIGEMPLTDSIL
CDGLTDAFHNCHMGITAENVAKKWQVSREDQDKVAVLSQNRTENAQKAG
HFDKEIVPVLVSTRKGLIEVKTDEFPRHGSNIEAMSKLKPYFLTDGTGT
VTPANASGINDGAAAVVLMKKSEADKRGLTPLARIVSWSQVGVEPSIMG
IGPIPAIKQAVTKAGWSLEDVDIFEINEAFAAVSAAIVKELGLNPEKVN
IEGGAIALGHPLGASGCRILVTLLHTLERMGRSRGVAALCIGGGMGIAM
CVQRE

An illustrative human ACAT2 gene sequence is provided by NCBI Genbank Accession Number NC_000006.12. An illustrative human ACAT2 nucleic acid sequence is provided by NCBI Genbank Accession Number NM_005891.3 and shown as SEQ ID NO: 4.

SEQ ID NO: 4
agcttgcaggcagcgcagggcagacggcggcaggagaagcaagatgaat
gcaggctcagatcctgtggtcatcgtctcggcggcgcggaccatcatag
gttccttcaatggtgccttagctgctgttcctgtccaggacctgggctc
cactgtcatcaaagaagtcttgaagagggccactgtggctccggaagat
gtgtctgaggtcatctttggacatgtcttggcagcaggctgtgggcaga
atcctgttagacaagccagtgtgggtgcaggaattccctactctgttcc
agcatggagctgccagatgatctgtgggtcaggcctaaaagctgtgtgc
cttgcagtccagtcaatagggataggagactccagcattgtggttgcag
gaggcatggaaaatatgagcaaggctcctcacttggcttacttgagaac
aggagtaaagataggtgagatgccactgactgacagtatactctgtgat
ggtcttacagatgcatttcacaactgtcatatgggtattacagctgaaa
atgtagccaaaaaatggcaagtgagtagagaagatcaggacaaggttgc
agttctgtcccagaacaggacagagaatgcacagaaagctggccatttt
gacaaagagattgtaccagttttggtgtcaactagaaaaggtcttattg
aagttaaaacagatgagtttcctcgccatgggagcaacatagaagccat
gtccaagctaaagccttactttcttactgatggaacgggaacagtcacc
ccagccaatgcttcaggaataaatgatggtgctgcagctgtcgttctta
tgaagaagtcagaagctgataaacgtgggcttacacctttagcacggat
agtttcctggtcccaagtgggtgtggagccttccattatgggaatagga
ccaattccagccataaagcaagctgttacaaaagcaggttggtcactgg
aagatgttgacatatttgaaatcaatgaagcctttgcagctgtctctgc
tgcaatagttaaagaacttggattaaacccagagaaggtcaatattgaa
ggaggggctatagccttgggccaccctcttggagcatctggctgtcgaa
ttcttgtgaccctgttacacacactggagagaatgggcagaagtcgtgg
tgttgcagccctgtgcattgggggtgggatgggaatagcaatgtgtgtt
cagagagaatgaattgcttaaactttgaacaacctcaatttctttttaa
actaataaagtactaggttgcaatatgtgaaatcagaggaccaaagtac
agatggaaaccatttcctacatcacaaaaacccaagtttacagcttgta
ctttactttaatgtgtaatactcaactcaaggtacaagacaattgcatt
taacattgttataaataaaaggaacatcagatcaatcattaagggctcc
agagtgaacagcatcttcataacttccatgtttatcatctttactttct
g

The ACAT inhibitor may be any entity which is capable of inhibiting the expression or activity of an ACAT in a cell.

The “expression” of an ACAT may refer to the level of transcription, translation i.e. mRNA and/or protein expression.

Measurement of the level or amount of a gene product may be carried out by any suitable method, for example including comparison of mRNA transcript levels, protein or peptide levels, between a treated cell and comparable cell which has not been treated according to the present invention.

The term “untreated cell” as defined herein would be a cell which had not been modified according to the present invention, e.g. to modulate the expression or activity of an ACAT protein or to modify the nucleic acid sequence of at least one gene encoding ACAT protein; and in which all other relevant features were the same.

The expression of specific genes encoding ACAT proteins can be measured by measuring transcription and/or translation of the gene. Methods for measuring transcription are well known in the art and include, amongst others, northern blot, RNA-Seq, in situ hybridization, DNA microarrays and RT-PCR. Alternatively, the expression of a gene may be measured indirectly by measuring the level of the gene product for example the protein encoded by said gene.

In some embodiments the expression of gene encoding an ACAT protein may decreased by at least 10%, at least 20%, at least 30%, at least 40%, at least 50%, at least 60%, at least 70%, at least 80%, at least 90%, at least 95% or 100% when compared to the expression of gene encoding an ACAT protein in a cell which has not been treated in accordance with the present invention.

By way of example, the ACAT inhibitor may be a small molecule inhibitor, a small inhibitory RNA (siRNA), a small hairpin RNA (shRNA), a micro RNA (miRNA), an antisense nucleic acid, or an anti-ACAT antibody or fragment thereof.

Suitably, the ACAT inhibitor is a pharmaceutically acceptable inhibitor.

Inhibiting the activity of an ACAT may mean that the ACAT inhibitor reduces enzymatic function of the ACAT. For example, the ACAT inhibitor may reduce the ability of an ACAT to convert free cholesterol to cholesteryl esters by at least 5%, at least 10%, at least 20%, at least 30%, at least 40%, at least 50%, at least 75%, at least 90% or at least 95% compared to the ability of an ACAT to convert free cholesterol to cholesteryl esters in the absence of the ACAT inhibitor.

The amount of cholesteryl ester produced from free cholesterol by an ACAT in the presence of the ACAT inhibitor may be at least 5%, at least 10%, at least 20%, at least 30%, at least 40%, at least 50%, at least 75%, at least 90% or at least 95% less than the amount of cholesteryl ester produced by the ACAT in a corresponding control assay in the absence of the ACAT inhibitor.

Assays for determining the conversion of free cholesterol to cholesteryl esters are known in the art (Lada et al; 2004; The Journal of Lipid Research, 45, 378-386.). By way of example, a rapid high-throughput assay for ACAT activity utilises a fluorescent sterol in which NBD-cholesterol (22-[N-(7-nitrobenz-2-oxa-1,3-diazol-4-yl)amino]-23,24-bisnor-5-cholen-3-ol) replaces the terminal segment of the alkyl tail of cholesterol to allow visualisation of its conversion to hydrophobic cholesterol esters in lipid droplets by ACAT activity within cells, resulting in an increase in cellular fluorescence (compared to the weak background fluorescence when NBD-cholesterol is in the polar environment of the cell membrane (Lada et al; 2004; The Journal of Lipid Research, 45, 378-386.). In addition, ACAT activity can for example be measured by the rate of [3H]cholesteryl oleate synthesis (e.g. Guo, JBC 2005; Chang, Biochemistry 1986) and measurement of the cholesterol/cholesteryl ester ratio (colorimetric or fluorometric methods). Moreover, unesterified cholesterol can be visualised by staining with filipin III from Streptomyces filipinensis and lipid droplets can be visualised by various methods, e.g. by LipidTox neutral lipid stain or Oil Red 0 staining.

Examples of small molecule inhibitors of ACAT include, but are not limited to, avasimibe, K604, rubimiallin, pactimibe, eflucimibe (F-12511), purpactins, manassantin A, diphenylpyridazine derivatives, glisoprenin A, beauveriolide I, beauveriolide III, U18666A, TMP-153, YM750, GERI-BP002-A, Sandoz Sah 58-035, VULM 1457, Lovastatin, 01976, CL-283,546, CI-999, E5324, YM17E, FR182980, ATR-101 (PD132301 or PD132301-2), F-1394, HL-004, cinnamic acid derivatives, cinnamic derivative, Dup 10 128, RP-73163, pyripyropene C, FO-1289, AS-183, SPC-15549, FO-6979, Angekica, ginseng, Decursin, terpendole C, beauvericin, spylidone, pentacecilides, CL-283,546, betulinic acid, shikonin derivatives, esculeogenin A, Wu-V-23, pyripyropene derivatives A, B, and D, glisoprenin B-D, saucerneol B, sespendole, diethyl pyrocarbonate, beauveriolide analogues, Acaterin, DL-melinamide, PD 138142-15, 0L277,082, EAB-309, Enniatin 15 antibiotics, Epi-cochlioquinone A, FCE-27677, FR186485, FR190809, NTE-122, obovatol, panaxadiols, protopanaxadiols, polyacetylenes, SaH 57-118, AS-186, BW-447A, 447C88, T-2591, TEI-6522, TEI-6620, XP 767, XR 920, GERI-BP001, gomisin N, gypsetin, helminthosporol, TS-962, isochromophilones, kudingosides, lateritin, naringenin, and MP-797.

Suitably, the ACAT inhibitor may be avasimibe or K604.

Avasimibe (CI-1011) is an orally bioavailable ACAT inhibitor. It was originally developed as an anti-hyperlipidemic drug, and was shown to significantly reduce plasma total triglyceride and VLDL-cholesterol. Avasimibe may be administered, for example, by once daily administration of 50-1000 mg/day, in particular 50-750 mg/day, or 50-500 mg/day.

K604 (4-[2-(1H-Benzimidazol-2-ylthio)ethyl]-N-[6-methyl-2,4-bis(methylthio)-3-pyridinyl]-1-piperazineacetamide dihydrochloride, 2-[4-[2-(Benzimidazol-2-ylthio)ethyl]piperazin-1yl]-N-[2,4-bis(methylthio)-6-methyl-3-pyridyl]acetamide dihydrochloride) is a selective ACAT1 inhibitor. Oral administration has shown promising results against macrophage foam cell formation and atherosclerosis progression in animal models.

The ACAT inhibitor may be a small inhibitory RNA (siRNA), a small hairpin RNA (shRNA), a micro RNA (miRNA), or an antisense nucleic acid targeted against a nucleic acid encoding an ACAT.

Inhibition (e.g. of the ACAT) may be achieved using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11: 429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al. (1998) Ann. Rev. Biochem. 67: 227-64). However, this response can be bypassed by using 21 nt siRNA duplexes (Elbashir et al. (2001) EMBO J. 20: 6877-88; Hutvagner et al. (2001) Science 293: 834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) noncoding RNAs that can effectively reduce the translation of target mRNAs by binding to their 3′ untranslated region

(UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art.

The ACAT inhibitor may be an antibody or an antigen-binding fragment thereof.

Suitably antibodies include, but are not limited to, monoclonal and polyclonal antibodies, engineered antibodies including chimeric, CDR-grafted and humanised antibodies, and artificially selected antibodies produced using phage display or alternative techniques.

Suitably the antibody is a monoclonal antibody.

Suitable antibody fragments capable of binding to a selected target, include Fv, ScFv, F(ab′) and F(ab′)₂. In addition, alternatives to classical antibodies may also be used in the invention, for example “avibodies”, “avimers”, “anticalins”, “nanobodies” and “DARPins”.

Antibodies capable of inhibiting ACAT activity may be identified using an ACAT activity as described herein.

The ACAT inhibitor or for use according to the present invention may have a direct antiviral activity against HBV and/or enhance humoral immunity to HBV.

A direct antiviral activity may refer to an ability of the ACAT inhibitor to reduce or inhibit HBV replication in a host organism/tissue/cell (i.e. the ACAT inhibitor is able to reduce or inhibit at least part of the HBV lifecycle in the absence of any further entities; such as host immune cells). For example, the ACAT inhibitor may directly reduce (i) HBV target cell entry, (ii) HBV genome replication, (iii) HBV viral capsid formation, (iv) HBV sAg assembly and/or (v) HBV virion and/or subviral particle release. Direct antiviral activity may be assessed using methods known in the art. For example, a cell line (e.g. HepG2-NTCP cells or primary human hepatocytes) may be infected with HBV and treated with the ACAT inhibitor (see e.g. present Example 6). Direct antiviral activity may be determined by a reduction of extracellular HBV DNA compared to a control experiment which has not been treated with the ACAT inhibitor. If the ACAT inhibitor can either reduce cccDNA and/or block HBsAg assembly/release, it may additionally reduce production/release of HBsAg.

Suitably, a direct antiviral activity may refer to an ability of the ACAT inhibitor to directly reduce HBV sAg assembly and/or directly reduce HBV virion and/or subviral particle release.

Enhanced humoral immunity to HBV may refer to an ability of the ACAT inhibitor to augment or increase the activity or function of components of a humoral immune response to HBV. Humoral immunity generally refers to an immune response that is mediated by macromolecules found in extracellular fluids such as secreted antibodies and complement proteins. In the context of the present invention, humoral immunity refers to an antibody-mediated immune response. Components of the immune system involved in promoting an antibody-mediated immune response against HBV include, but are not limited to, B-cells and CD4+ T cells, such as CD4⁺ T_{FH} cells.

Accordingly, the ACAT inhibitor for use in the present invention may enhance the activity of B cells and/or CD4⁺ T cells. Suitably, enhancing B cell activity enhances humoral immunity to HBV. This activity may be particularly advantageous, for example, when the ACAT inhibitor is administered as part of—or in combination with—a therapeutic vaccine as described herein.

The activity of B cells may be determined using methods which are known in the art. For example, B cell activity may be assessed by determining calcium mobilisation in B cells following B-cell receptor engagement (see e.g. present Example 4). Enhancement of B cell activity may be determined by an increase in calcium mobilisation in B cells compared to a control experiment which has not been treated with the ACAT inhibitor. An alternative readout for B cell signalling is phosphoflow for downstream signalling molecules. The activity of B cells is more commonly measured by determining antibody (e.g. IgG or IgM) production following differentiation into plasma cells using ELISPOT or ELISA assays; enhancement of plasma cell antibody production may thus be measured upon addition of an ACAT inhibitor. Alternatively, enhancement of B cell activity may be determined by an increase in production of effector cytokines with anti-HBV activity (e.g. percentage of IFNγ or IL-6 positive cells) as determined by flow cytometry or amount of effector cytokine mRNA or protein as determined by qPCR, ELISA or ELISPOT, respectively) compared to a control experiment which has not been treated with the ACAT inhibitor. B cell activity may also be assessed by determining expansion or activation or reduction in exhaustion of global or memory or HBs-Ag-specific B cells following treatment with an ACAT inhibitor using methods which are known in the art.

For example, calcium mobilisation in B-cells treated with an ACAT inhibitor may be increased by at least 10%, at least 20%, at least 30%, at least 40%, at least 50%, at least 75% or at least 100% compared to control cells which have not been treated with the ACAT inhibitor.

The activity of CD4⁺ T cells may be determined using methods which are known in the art. For example, CD4⁺ T cell activity may be assessed by determining effector cytokine production (i.e. IFNγ, IL-21 or IL-4) following treatment with the ACAT inhibitor (see e.g. present Example 1). Enhancement of CD4⁺ T cell activity may be determined by an increase in effector cytokine (e.g. percentage of effector cytokine positive cells as determined by flow cytometry or amount of effector cytokine mRNA or protein as determined by qPCR, ELISA or ELISPOT, respectively) compared to a control experiment which has not been treated with the ACAT inhibitor.

For example, levels of effector cytokine in CD4⁺ T cell treated with an ACAT inhibitor may be increased by at least 10%, at least 20%, at least 30%, at least 40%, at least 50%, at least 75% or at least 100% compared to control cells which have not been treated with the ACAT inhibitor.

Suitably, the CD4+ T cell may be a CD4⁺ T_{FH} cell. A CD4⁺ T_{FH} cell may be a CD3+CD4+CXCR5+PD-1+ cell. The activity of CD4⁺ T_{FH} cells may be assessed by determining, for example, the expression/production of CD4OL, 4-1BB, ICOS, OX40, IL-21 and/or IL-4. Expression of CD40-L and OX40 in particular can be used to delineate antigen-activated cells.

The ACAT inhibitor for use according to the present invention may: a) exhibit direct antiviral activity against HBV; b) enhance humoral immunity to HBV; and c) enhance T cell immunity to HBV.

Suitably, enhanced T cell immunity to HBV may refer to CD8+ T cell activity. The activity of CD8+ T cells may be determined using methods which are known in the art. For example, CD8+ T cell activity may be assessed by determining effector cytokine production (e.g. IFNγ/TNF/IL-2) or cytotoxicity (measured by flow cytometric evaluation of CD107a expression as a marker of degranulation and/or expression of cytotoxic mediators perforin, granzyme, and/or by quantification of specific target cell lysis by e.g. ToxiLight Cytotoxicity Assay), upon stimulation with viral or HCC peptides or mitogens, following treatment with the ACAT inhibitor (see e.g. present Example 1 and Example 5). Enhancement of CD8⁺ T cell activity may be determined by an increase in effector function (e.g. percentage of effector cytokine or degranulating or cytotoxic mediator positive cells as determined by flow cytometry or amount of effector cytokine mRNA or protein as determined by qPCR or ELISPOT, respectively) compared to a control experiment which has not been treated with the ACAT inhibitor. Proliferative expansion of antigen-specific CD8 T cells can also be assessed by staining with MHC/peptide multimers.

For example, levels of effector cytokine in CD8⁺ T cells and/or number of CD8⁺ T cells able to produce detectable effector cytokines treated with an ACAT inhibitor may be increased by at least 10%, at least 20%, at least 30%, at least 40%, at least 50%, at least 75% or at least 100% compared to control cells which have not been treated with the ACAT inhibitor.

Enhancing humoral and/or T cell immunity against HBV may be efficacious in restoring a productive immune response against HBV in a subject. For example, enhancing humoral and/or T cell immunity against HBV may restore an exhausted immune response against HBV in a subject.

### Therapeutic Vaccine

A therapeutic vaccine refers to a vaccine which is administered to a subject already suffering from a disease, in order to treat said existing disease.

In the context of the present invention, a therapeutic vaccine is administered to a subject with existing HBV infection in order to enhance the subject's immune response to the HBV.

Suitably, the ACAT inhibitor may be included in a therapeutic vaccine composition which further comprises HBV immunogens.

Suitable HBV immunogens include, but are not limited to, whole HBV antigens such as HBV core, polymerase or surface antigen (these may be provided as a vector-encoded nucleic acid vaccine [e.g. adenoviral] or as protein, in each case the HBV antigen may be administered as a prime dose followed by an MVA boost), T cell epitope(s) from HBcAg, polymerase or surface antigen and HBV DNA.

Suitably, the therapeutic vaccine will be formulated for administration by injection, for example by intramuscular, intradermal, intravenous or sub-cutaneous injection.

A therapeutic vaccine will generally be administered in admixture with a pharmaceutical carrier, excipient or diluent, particularly for human therapy.

Suitably, the therapeutic vaccine composition may comprise a nucleic acid construct encoding an ACAT inhibitor. By way of example, the nucleic acid construct may encode a small inhibitory RNA (siRNA), a small hairpin RNA (shRNA), a micro RNA (miRNA), or an antisense nucleic acid as described herein.

### Combination

The ACAT inhibitor for use according to the present invention may be administered in combination with a further pharmaceutically active agent.

Suitably, “in combination” may mean that the ACAT inhibitor and the further pharmaceutically active agent are administered to the subject in a simultaneous, combined, sequential or separate manner.

By “simultaneous”, it is to be understood that the two agents are administered concurrently, whereas the term “combined” is used to mean they are administered, if not simultaneously, then “sequentially” within a time frame that they both are available to act therapeutically within the same time frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction there-between, and their respective half-lives.

In contrast to “combined” or “sequential”, “separate” may be understood as meaning that the gap between administering one agent and the other agent is significant, i.e. the first administered agent may no longer be present in the bloodstream in a therapeutically effective amount when the second agent is administered.

Suitably, the ACAT inhibitor and further pharmaceutically active agent are administered in a simultaneous, combined, or sequential manner to a subject.

Suitably, the ACAT inhibitor and further pharmaceutically active agent may be administered as a single composition. For example, the ACAT inhibitor may comprise a nucleic acid construct encoding a siRNA, a shRNA, a miRNA, or an antisense nucleic acid targeted against a nucleic acid encoding an ACAT—as described herein—and be provided as part of a therapeutic vaccine as described herein.

Suitably, the ACAT inhibitor may be administered as a separate composition to the further pharmaceutically active agent. For example, the ACAT inhibitor may be a small molecule (e.g. avasimibe or K604) administered orally and the further pharmaceutically active agent may be a therapeutic HBV vaccine administered by intramuscular, subcutaneous or intradermal or intravenous injection. Alternatively, the ACAT inhibitor may a small molecule (e.g. avasimibe or K604) administered orally and the further pharmaceutically active agent may be an antiviral (e.g. a nucleoside/nucleotide analogue).

The further pharmaceutically active agent may be selected from the group consisting of an antiviral, a therapeutic HBV vaccine, an immunostimulatory cytokine, a checkpoint inhibitor, TCR-gene-engineered cells, activators of innate immunity, monoclonal or bispecific antibodies, CAR cells, soluble T cell receptors, or any combination thereof.

Antivirals include, but are not limited to, nucleoside/nucleotide analogues (NAs or NUCs). Suitable NAs include Tenofovir, Entecavir, Lamivudine, Telbivudine and Adefovir. Antivirals currently in use include pegylated interferon-alpha. Further antivirals include agents which interfere or inhibit the ability of the HBV to enter host cells, such as Myrcludex B and others such as capsid assembly inhibitors, anti-HBV siRNA, etc

Current NA are generally not considered to be capable of reducing HBV cccDNA and/or blocking HBsAg assembly/release, and therefore do not reduce production/release of HBsAg.

Suitable immunostimulatory cytokines include, but are not limited to, IFN-α, pegylated IFN-α, IL-2 and IL-2 variants, IFNγ, GM-CSF, IL-7, IL-12, IL-15, IL-18 and IL-21.

Suitable checkpoint inhibitors include, but are not limited to, both inhibitory and activatory molecules, and interventions may apply to either or both types of molecule. Immune checkpoint inhibitors include, but are not limited to, PD-1 inhibitors, PD-L1 inhibitors, Lag-3 inhibitors, Tim-3 inhibitors, TIGIT inhibitors, BTLA inhibitors and CTLA-4 inhibitors, for example. Co-stimulatory antibodies deliver positive signals through immune-regulatory receptors including but not limited to ICOS, CD137, CD27, OX-40 and GITR.

Suitably, the checkpoint inhibitor may be a PD-1 or PD-L1 inhibitor.

Examples of suitable immune checkpoint interventions which prevent, reduce or minimize the inhibition of immune cell activity include pembrolizumab, nivolumab, atezolizumab, durvalumab, avelumab, tremelimumab and ipilimumab.

A TCR-gene-engineered cell refers to a cell, preferably a T cell, which has been engineered to introduce an exogenous nucleic acid encoding an exogenous TCR into the cell and thus redirect the specificity of the cell to a target antigen of interest. In the context of the present invention, the cell is suitably redirected to an HLA and HBV-peptide complex of interest. TCR-gene-engineered cells and anti-HBV T cells are described in art, for example see Gehring et al. (J Hepatol. 2011 Jul;55(1):103-10); Qasim et al. (J Hepatol. 2015 Feb;62(2):486-91) and Tan et al. (Gastroenterology. 2019 May;156(6):1862-1876.e9); each of which is incorporated herein by reference.

The present invention also encompasses administering an ACAT inhibitor in combination with HBV-specific T cells which have been isolated, and preferably enriched, from a subject. The subject is suitably the same subject with HBV infection who is to be treated according to the invention.

Soluble T cell receptors (sTCR) typically comprise the variable domain and at least part of the TCR constant domain but lack the transmembrane domain and intracellular, cytoplasmic domain. Suitably, the sTCR does not comprise a transmembrane domain. Suitably, the sTCR is not anchored on the surface of cell. In the context of the present invention, the sTCR is directed to a HBV target antigen of interest.

A classical CAR is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

When the CAR binds the target-antigen, this results in the transmission of an activating signal to the T-cell it is expressed on. Thus the CAR directs the specificity and cytotoxicity of the T cell towards cells expressing the targeted antigen.

CARs typically therefore comprise: (i) an antigen-binding domain; (ii) a spacer; (iii) a transmembrane domain; and (iii) an intracellular domain which comprises or associates with a signalling domain.

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example, the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell. The antigen binding domain may be based on a natural ligand of the antigen.

The antigen binding domain may comprise an affinity peptide from a combinatorial library or a de novo designed affinity protein/peptide.

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

The transmembrane domain is the portion of the CAR which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the CAR. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

The transmembrane domain may be derived from CD28, which gives good receptor stability.

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain may comprise:
(i) an ITAM-containing endodomain, such as the endodomain from CD3 zeta; and/or
(ii) a co-stimulatory domain, such as the endodomain from CD28; and/or
(iii) a domain which transmits a survival signal, for example a TNF receptor family endodomain such as OX-40 or 4-1BB.

Illustrative HBV-specific CARs are described in Festag et al. (Mol Ther. 2019 May 8;27(5):947-959) and Krebs et al. (Gastroenterology. 2013 Aug;145(2):456-65)—each of which is incorporated herein by reference.

Suitably, the CAR cell may be a T cell comprising an exogenous nucleic acid construct which encodes a CAR as described herein.

In one aspect the present invention provides a TCR-gene-engineered cell or CAR cell having reduced ACAT activity and/or reduced ACAT expression for use in the treatment of HBV infection in a subject.

ACAT activity and/or expression may be reduced, for example, by an in vitro treatment of the TCR-gene-engineered cell or CAR cell with an ACAT inhibitor prior to administration to the subject.

For example, the TCR-gene-engineered cell or CAR cell may be engineered ex vivo or in vitro to comprise a nucleic acid construct which is capable of inhibiting ACAT expression or translation. By way of example, the nucleic acid construct may encode a siRNA, a shRNA, a miRNA, or an antisense nucleic acid targeted against a nucleic acid encoding an ACAT as described herein.

Suitably, the nucleic acid construct may comprise a first nucleic acid sequence encoding an exogenous TCR or a CAR and a second nucleic sequence encoding a siRNA, a shRNA, a miRNA, or an antisense nucleic acid targeted against a nucleic acid encoding an ACAT.

ACAT activity and/or expression may be reduced, for example, by administering the TCR-gene-engineered cell or CAR cell to the subject in combination with an ACAT inhibitor.

For example, the ACAT inhibitor may be administered as a separate composition to the TCR-gene-engineered cell or CAR cell. For example, the ACAT inhibitor may a small molecule (e.g. avasimibe or K604) administered orally and the TCR-gene-engineered cell or CAR cell may be administered as a composition by intravenous, intramuscular, subcutaneous or intradermal injection.

Suitably, the invention may comprise reducing ACAT activity and/or expression in the TCR-gene-engineered cell or CAR cell by an in vitro treatment with an ACAT inhibitor prior to administration to the subject; and subsequently administering the TCR-gene-engineered cell or CAR cell to the subject in combination with an ACAT inhibitor (e.g. a small molecule ACAT inhibitor).

Methods for introducing an exogenous TCR or CAR into a cell are well known in the art and include introducing into a cell in vitro or ex vivo a polynucleotide encoding a TCR or CAR as defined herein. Suitably, the method further comprises incubating the cell under conditions causing expression of TCR or CAR. Optionally, the method may further comprise a step of purifying the engineered cells. The nucleic acid molecule encoding the TCR or CAR may be introduced into the cell using known vectors, for example a lentiviral vector. The term “vector” includes an expression vector, i.e. a construct capable of in vivo or in vitro/ex vivo expression. Also encompassed are cloning vectors.

Viral delivery systems include but are not limited to adenovirus vector, an adeno-associated viral (AAV) vector, a herpes viral vector, retroviral vector, lentiviral vector, baculoviral vector.

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses. A detailed list of retroviruses may be found in Coffin et al (“Retroviruses” 1997 Cold Spring Harbour Laboratory Press Eds: J M Coffin, S M Hughes, H E Varmus pp 758-763). Lentiviruses also belong to the retrovirus family, but they can infect both dividing and non-dividing cells (Lewis et al (1992) EMBO J. 3053-3058). The vector may be a retroviral vector. The vector may be based on or derivable from the MP71 vector backbone. The vector may lack a full-length or truncated version of the Woodchuck Hepatitis Response Element (WPRE). For efficient infection of human cells, viral particles may be packaged with amphotropic envelopes or gibbon ape leukemia virus envelopes.

Suitably, a TCR-gene-engineered cell or CAR cell as described herein may be treated in vitro or ex vivo with an ACAT inhibitor according to the present invention. Following in vitro or ex vivo treatment with the ACAT inhibitor, the TCR-gene-engineered cell or CAR cell may subsequently be administered to a subject to treat HBV infection as described herein. The subject may be at risk of developing HBV-associated hepatocellular carcinoma (HCC), may have HBV-associated HCC or may have previously had HBV-associated HCC. Thus, following in vitro or ex vivo treatment with the ACAT inhibitor, the TCR-gene-engineered cell or CAR cell may subsequently be administered to a subject to treat HCC as described herein.

Accordingly, in one aspect the present invention provides a method of treating HBV infection in a subject which comprises the following steps:
(i) isolation of a cell-containing sample from a subject;
(ii) introducing a nucleic acid sequence encoding a TCR or CAR to the cells; and
(iii) administering the cells from (ii) to the subject.

Suitably the cells from (ii) may be expanded in vitro before administration to the subject.

The subject in steps (i) and (iii) may be the same or a different subject. In other words, the cells may be autologous or may be allogenic to the subject to be treated.

Step (ii) may optionally also comprise introducing nucleic acid construct which is capable of inhibiting ACAT expression or translation.

Suitable activators of innate immunity include, but are not limited to, toll-like receptor (TLR) agonists and RIG-I/NOD agonists. Examples of TLR agonists include TLR-7 agonists, TLR-8 agonists and TLR-9 agonists. These agonists are typically provided as small molecules in a composition for oral administration.

### Hepatitis B Virus (HBV) Infection

HBV infection remains a major health problem worldwide with an estimated 350 million chronic carriers. Approximately 25% of carriers die from chronic hepatitis, cirrhosis, or liver cancer.

The hepatitis B virus (HBV) is an enveloped, partially double-stranded DNA virus. The compact 3.2 kb HBV genome consists of four overlapping open reading frames (ORF), which encode for the core, polymerase (Pol), envelope and X-proteins. The Pol ORF is the longest and the envelope ORF is located within it, while the X and core ORFs overlap with the Pol ORF. The lifecycle of HBV has two main events: 1) generation of covalently closed circular DNA (cccDNA) from relaxed circular (RC DNA), and 2) reverse transcription of pregenomic RNA (pgRNA) to produce RC DNA. Prior to the infection of host cells, the HBV genome exists within the virion as RC DNA. HBV virions are able to gain entry into host cells by non-specifically binding to the negatively charged proteoglycans present on the surface of human hepatocytes and via the specific binding of HBV surface antigens (HBsAg) to the hepatocyte sodium-taurocholate cotransporting polypeptide (NTCP) receptor. All HBV viral mRNAs are capped and polyadenylated, and then exported to the cytoplasm for translation. In the cytoplasm, the assembly of new virons is initiated and nascent pgRNA is packaged with viral Pol so that reverse transcription of pgRNA, via a single stranded DNA intermediate, into RC DNA can commence.

The secretion of antiviral cytokines in response to HBV infection by the hepatocytes and/or the intra-hepatic immune cells plays a central role in the viral clearance of the infected liver.

However, chronically infected patients only display a weak immune response due to various escape strategies adopted by the virus to counteract the host cell recognition systems and the subsequent antiviral responses. Many observations showed that several HBV viral proteins could counteract the initial host cellular response by interfering with the viral recognition signalling system and subsequently the interferon (IFN) antiviral activity. Among these, the excessive secretion of HBV empty sub-viral particles (SVPs, HBsAg) are thought to participate to the maintenance of the immunological tolerant state observed in chronically infected patients (CHB). The persistent exposure to MHC/peptide complexes processed from HBsAg and other viral antigens can lead to HBV-specific T-cell deletion or to progressive functional impairment. Moreover, HBsAg may suppress the function of immune cells such as monocytes, dendritic cells (DCs) and natural killer (NK) cells by direct interaction. In addition HBsAg may drive exhaustion of the antigen-specific B cell response by binding to their cognate B cell receptor (comprising anti-HBs antibody).

HBsAg quantification is the principle biomarker for prognosis and treatment response in functional cure of chronic hepatitis B. However, the achievement of HBsAg loss and seroconversion is rarely observed in chronically infected patients but remains one of the ultimate goals of therapy.

Hepatitis B e-antigen (also called HBeAg) is a viral protein that is secreted by hepatitis B infected cells. HBeAg is associated with chronic hepatitis B infections and is used as a marker of active viral disease and a patient's degree of infectiousness.

The function of the hepatitis B virus precore or HBeAg is not completely known. However, HBeAg is postulated to play a key role in viral persistence. HBeAg is thought to promote HBV chronicity by functioning as an immunoregulatory protein. In particular, the HBeAg is a secreted accessory protein, which appears to attenuate the host immune response to the intracellular nucleocapsid protein. The HBeAg acts as an immune tolerogen contributing to

HBV persistence, and possibly functions in utero considering that soluble HBeAg traverses the placenta. Furthermore, HBeAg downregulates: i) cellular genes controlling intracellular signaling; and ii) the Toll-like receptor 2 (TLR-2) to dampen the innate immune response to viral infection. In the absence of HBeAg, HBV replication is associated with an upregulation of the TLR2 pathway.

Laboratory diagnosis of hepatitis B infection focuses on the detection of the hepatitis B surface antigen, HBsAg.

Acute HBV infection may be characterized by the presence of HBsAg and immunoglobulin M (IgM) antibody to the core antigen, HBcAg. During the initial phase of infection, patients are also seropositive for hepatitis B e antigen (HBeAg). HBeAg is usually a marker of high levels of replication of the virus. The presence of HBeAg indicates that the blood and body fluids of the infected individual are highly infectious.

The World Health Organization provides that chronic HBV infection may be characterized by the persistence of HBsAg for at least 6 months (with or without concurrent HBeAg).

Persistence of HBsAg is the principal marker of risk for developing chronic liver disease, liver cirrhosis and liver cancer (hepatocellular carcinoma) later in life.

Suitably, the subject may have chronic HBV infection characterised by a viral load >2000 IU/ml and liver inflammation.

Suitably, the subject may be a subject with early chronic HBV infection. For example, the subject may be a neonate or paediatric subject with a maternally transmitted HBV infection.

### HBV Genotypes

HBV is differentiated into many genotypes, according to genome sequence. To date, eight well-known genotypes (A-H) of the HBV genome have been defined. Moreover, two new genotypes, I and J, have also been identified.

Some HBV genotypes are further classified as sub-genotypes. HBV sequence is characterized by >8% nucleotide differences for genotype, and 4%-8% nucleotide differences for sub-genotype.

Different genotypes and sub-genotypes show different geographical distribution, and are related to disease progression, clinical progression, response to antiviral treatment, and prognosis. A-D and F genotypes are divided into various sub-genotypes; no sub-genotypes have been defined for E, G and H genotypes. Genotype A is widespread in sub-Saharan Africa, Northern Europe, and Western Africa; genotypes B and C are common in Asia; genotype C is primarily observed in Southeast Asia; genotype D is dominant in Africa, Europe, Mediterranean countries, and India; genotype G is reported in France, Germany, and the United States; and genotype H is commonly encountered in Central and South America. Genotype I has recently been reported in Vietnam and Laos. The newest HBV genotype, genotype J, has been identified in the Ryukyu Islands in Japan.

Suitably, the HBV infection may be caused by an HBV genotype with a high tendency to cause chronic HBV infection. For example, the infection may be cause by HBV genotype A or C.

Suitably, the infection is caused by HBV genotype C or D.

Suitably, the infection is caused by HBV genotype C.

### HBV-Associated Hepatocellular Carcinoma (HCC)

HCC usually occurs in the setting of chronic liver inflammation, and is most closely linked to chronic viral hepatitis infection.

Chronic HBV infection accounts for at least 50% cases of HCC worldwide. Both indirect and direct mechanisms are involved in HCC oncogenesis by HBV. HCC-promoting HBV factors include long-lasting infection, high levels of HBV replication, HBV genotype, HBV integration, specific HBV mutants, and HBV-encoded oncoproteins (e.g., HBx and truncated preS2/S proteins). Recurrent liver inflammation caused by host immune responses during chronic HBV infection can lead to liver fibrosis and cirrhosis and accelerate hepatocyte turnover rate and promote accumulation of mutations.

An ACAT inhibitor for use according to the present invention may provide a range of advantageous effects for a subject at risk of developing HBV-associated HCC or a subject with HBV-associated HCC. For example, ACAT inhibition may provide advantageous effects relating to anti-carcinogenic, immune-boosting, synergistic effect with other cancer immunotherapies, and/or suppressing ongoing HBV replication.

A subject at risk of HCC may be a subject with HBV-related cirrhosis, a subject with HBV-related pre-cancerous changes.

A subject with HCC may be a subject requiring resection or other therapies including immunotherapies for HCC.

The subject with HCC may have been determined to be unsuitable for other therapeutic approaches e.g. due to advanced tumour stage (e.g. BCLC C+D), severe liver dysfunction (e.g. Child Pugh C), reduced performance status (e.g. determined by ECOG, WHO), severe side effects of treatment, patient's preference of oral treatment, or other comorbidities.

The subject may have previously been successfully treated for HCC but be at risk of HCC recurrence or metastasis.

### Treatment

The term “treat/treatment/treating” may refer generally to administering a medicament or pharmaceutical composition of the invention to a subject having an existing disease or condition in order to lessen, reduce or improve at least one symptom associated with the disease and/or to slow down, reduce or block the progression of the disease.

Suitably, the present invention provides a curative treatment. As used herein, the term “curative treatment” is intended to refer to a functional cure of HBV infection or a partial cure of HBV infection.

A functional cure of HBV infection may be determined using laboratory tests for HBV markers, such as HBsAg and/or HBV DNA. Suitable methods include those provided by

Abbvie Architect and Roche Taqman.

A functional cure of HBV infection may be defined as sustained off-treatment viral suppression and HBsAg loss in the subject (suitably in a sample from the subject, preferably wherein the sample is a blood sample), optionally with anti-HBs antibody seroconversion.

A partial cure of HBV infection may be defined by sustained off-treatment levels of HBV DNA of <20001 U/ml in a sample (e.g. a blood sample) from a subject following treatment according to the present invention.

A partial functional cure of HBV infection may be determined by a 1log or greater reduction in HBsAg in a sample (e.g. a blood sample) from a subject following a treatment according to the present invention compared to prior to treatment.

A functional cure of HBV infection may be determined by levels of HBsAg and/or HBV DNA below the lower limit of detection in a sample (e.g. a blood sample) from a subject who has previously been determined to have HBV infection (i.e. prior to treatment according to the invention). By way of example a level of <0.02 IU/ml for HBsAg (Abbvie Architect) and/or <20 IU/ml for HBV DNA (Roche Taqman) may be used to determine a functional cure of HBV infection.

“HBV load” as used herein may refer to detection of HBV DNA in a sample (e.g. a blood sample) from a subject as described herein.

### Dosage

The skilled person can readily determine an appropriate dose of one of the agents of the invention to administer to a subject without undue experimentation. Typically, a physician will determine the actual dosage which will be most suitable for an individual patient and it will depend on a variety of factors including the activity of the specific agent employed, the metabolic stability and length of action of that agent, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

The skilled person appreciates, for example, that route of delivery (e.g. oral vs. intravenous vs. subcutaneous etc.) may impact the required dosage (and vice versa). For example, where particularly high concentrations of an agent within a particular site or location are desired, focussed delivery may be preferred. Other factors to be considered when optimizing routes and/or dosing schedule for a given therapeutic regimen may include, for example, the disease being treated (e.g. type or stage etc.), the clinical condition of a subject (e.g. age, overall health etc.), the presence or absence of combination therapy, and other factors known to medical practitioners.

The dosage is such that it is sufficient to improve symptoms or markers of the disease—as described herein.

### Subject

A “subject” refers to either a human or non-human animal.

Examples of non-human animals include vertebrates, for example mammals, such as non-human primates (particularly higher primates),

Preferably, the subject is a human.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

### ACAT Inhibition Enhances HBV-Specific CD8+ and CD4+ T Cell Responses, Including Intrahepatic and Intratumoural CD8+ T Cell Responses

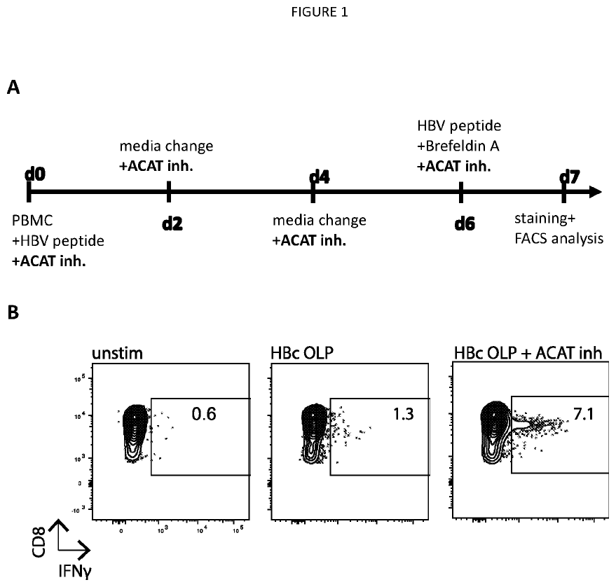

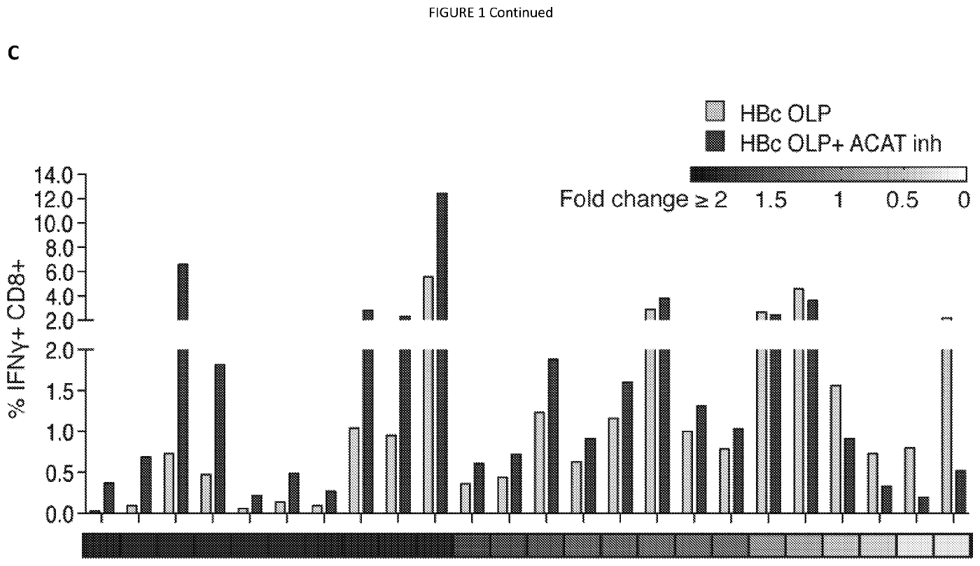

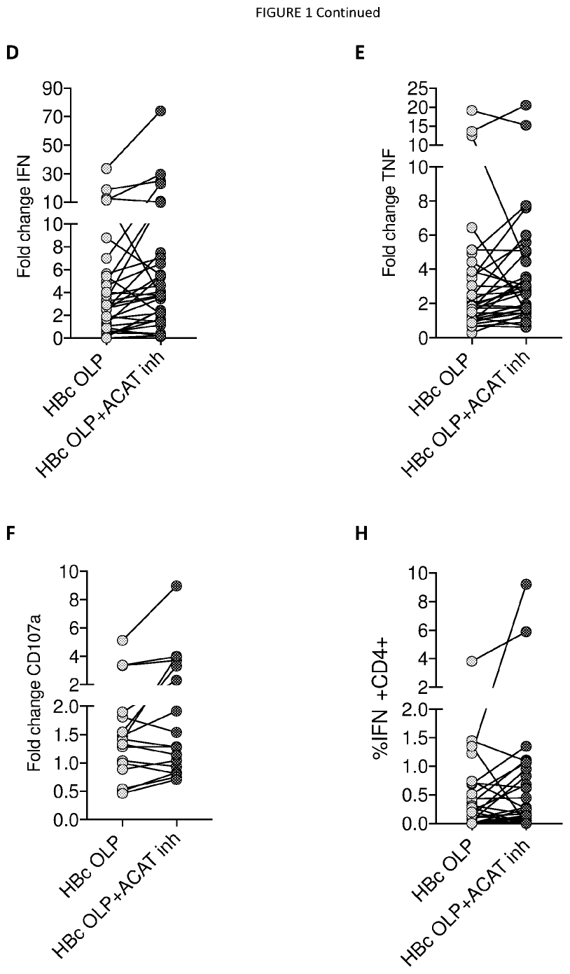

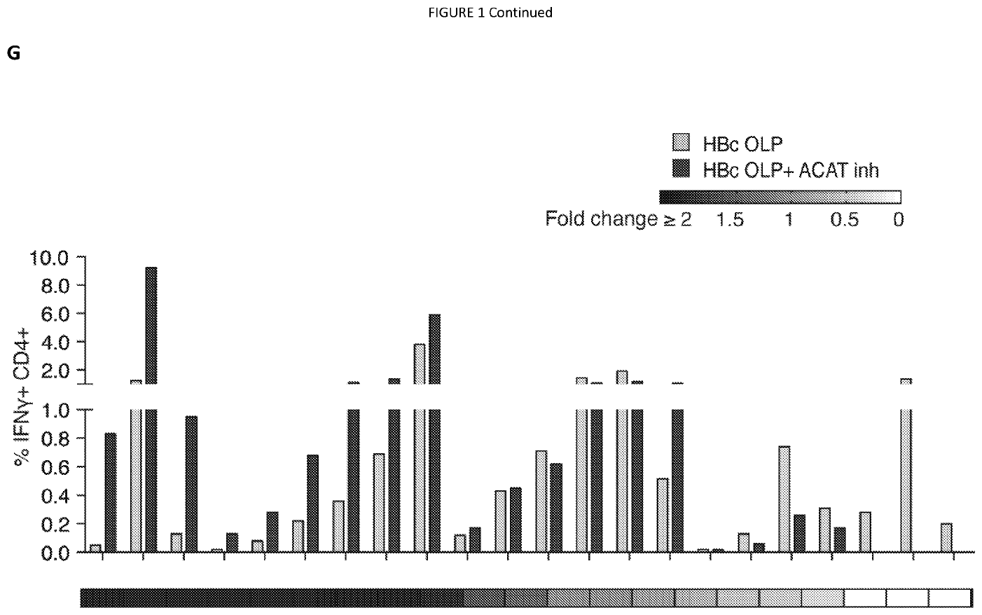

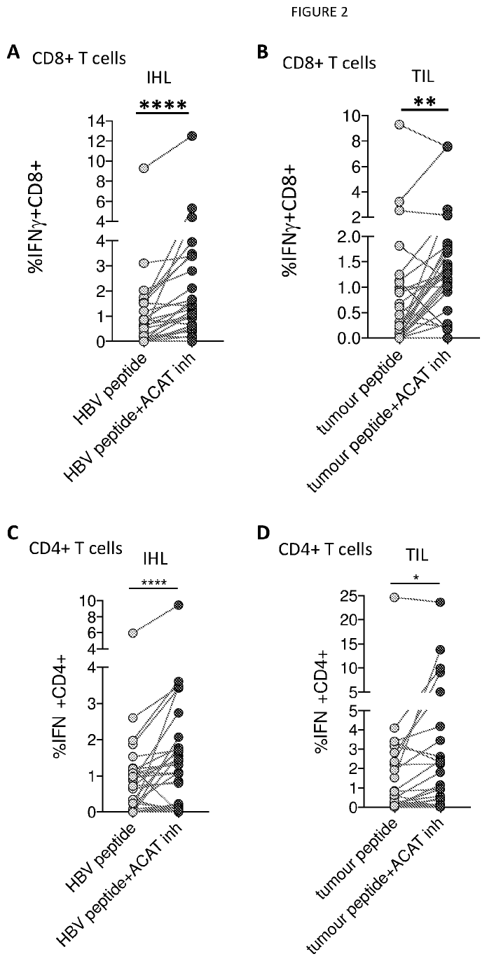

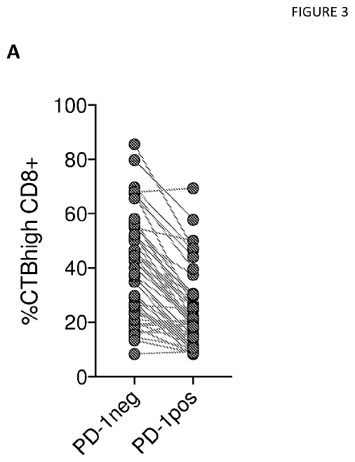

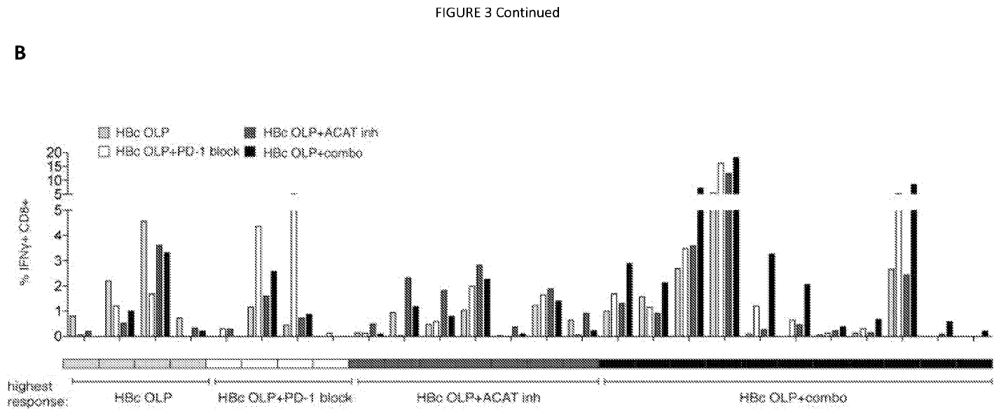

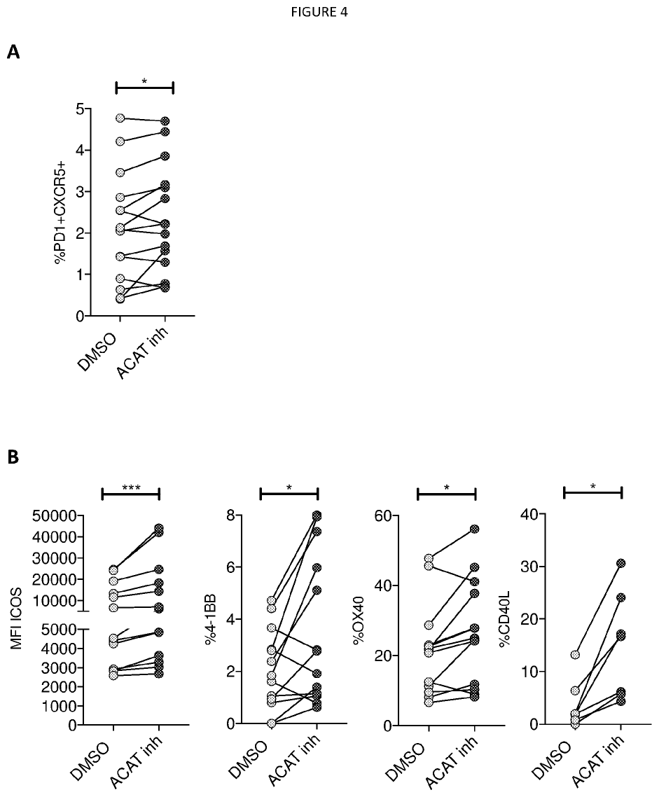

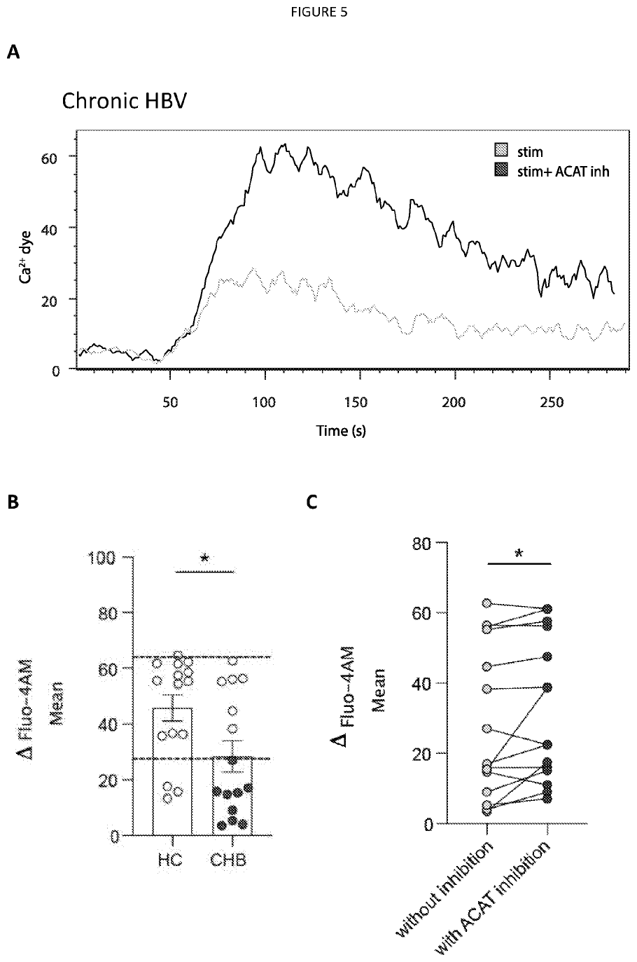

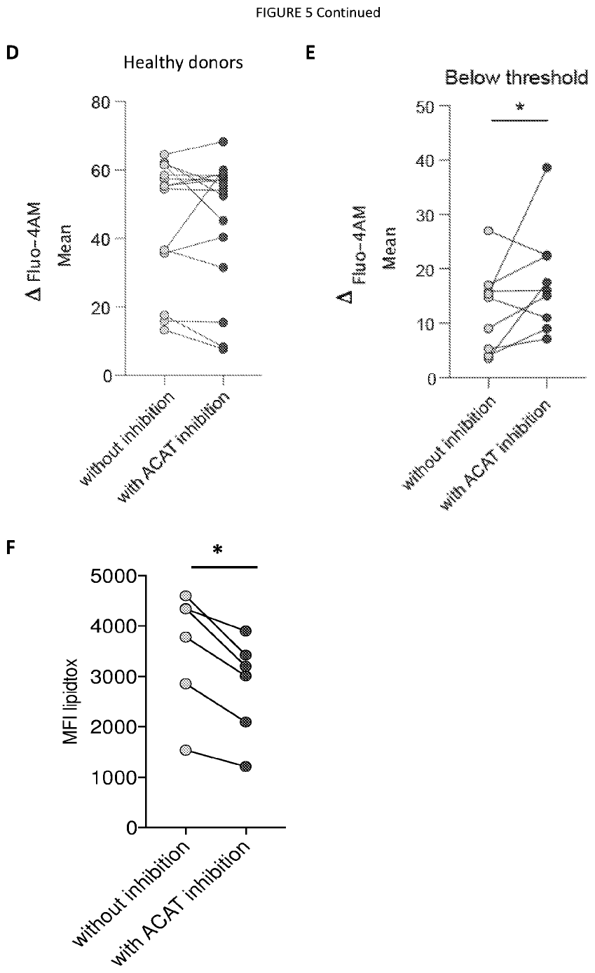

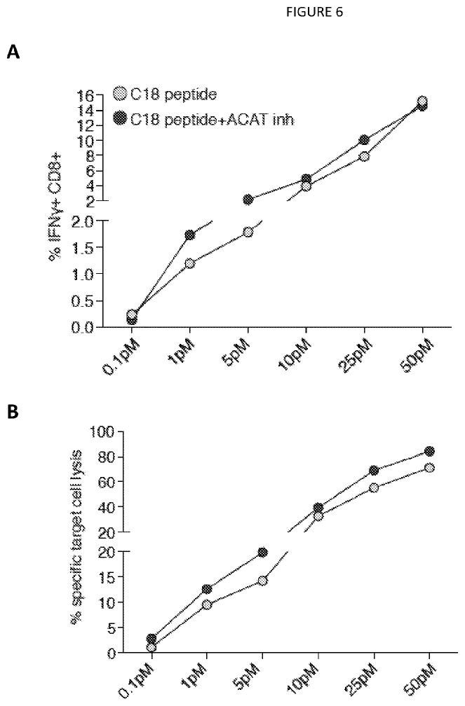

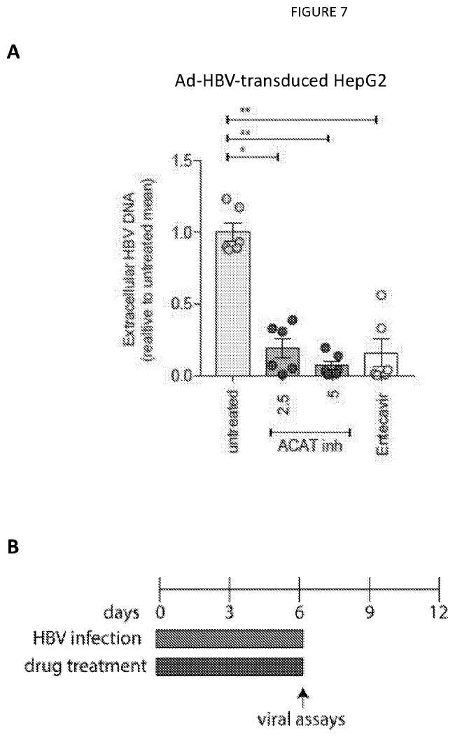

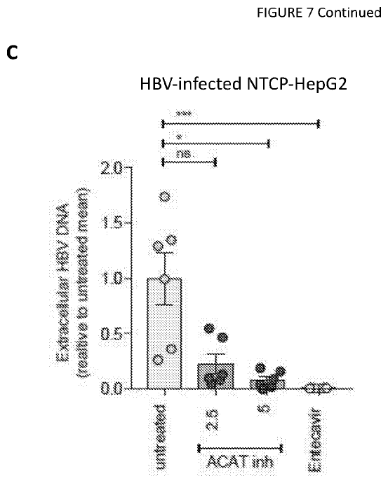

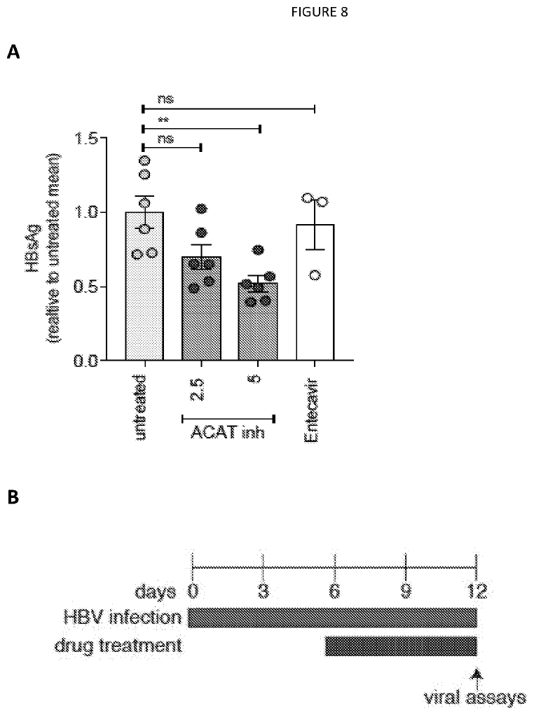

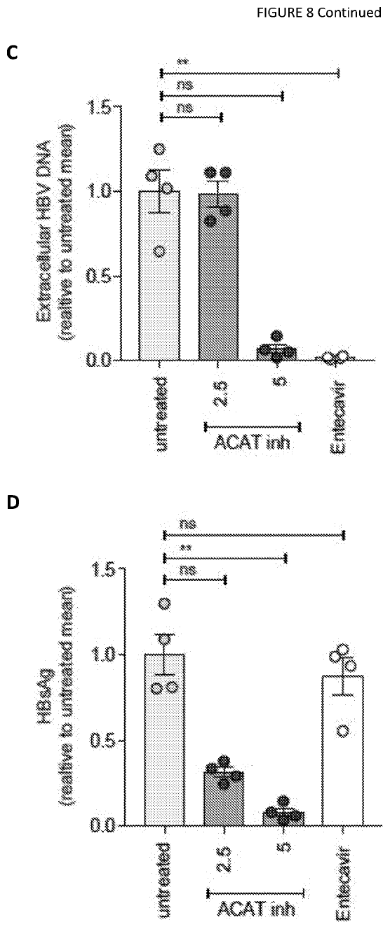

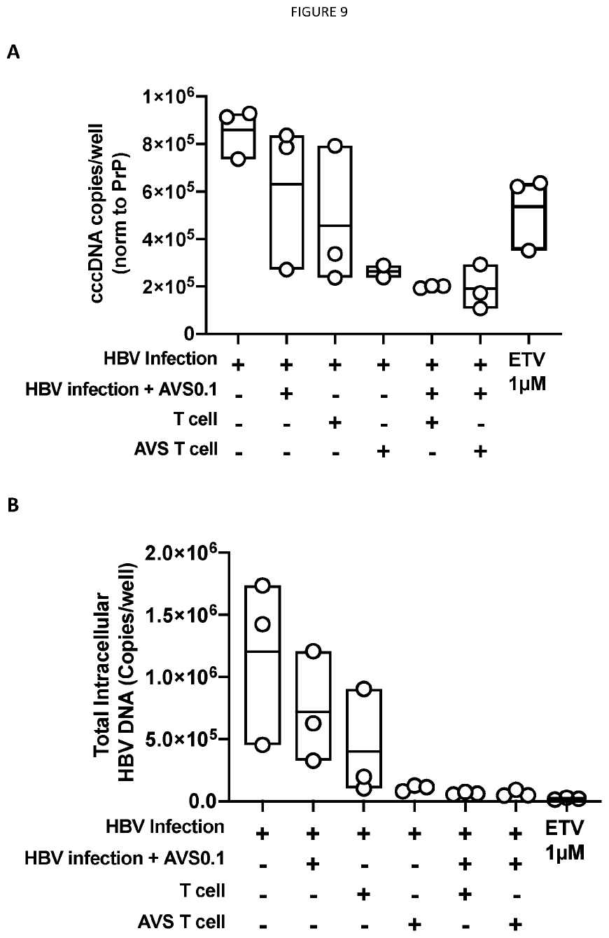

HBV-specific T cells are highly exhausted with a limited antiviral function (e.g. shown by limited cytokine production like IFNγ, TNF and limited cytotoxic function shown by limited CD107 mobilization to the cell surface). ACAT inhibition enhances HBV-specific CD8+ (see FIG. 1B-F) and CD4+ T cell responses (see FIG. 1G-H) from lymphocytes isolated from the blood of patients with chronic HBV infection.

ACAT inhibition also enhances HBV-specific T cell responses of liver-infiltrating lymphocytes isolated from patients with chronic HBV infection (see FIG. 2A and FIG. 2C).

Tumours express peptides (TAA peptides) that can be recognized by antitumoural T cells which ideally results in a lysis of tumour cells. In HBV-associated liver cancer (HCC) HBV peptides can also act as tumour antigens. ACAT inhibition enhances tumour-specific (HCC-specific) T cell responses of tumour-infiltrating lymphocytes isolated from human liver cancer tissue (see FIG. 2B and FIG. 2D). This includes HBV-specific T cells in HBV-associated liver cancer.

ACAT inhibition increases expansion of HBV-specific CD8+ T cells, lipid raft formation, TCR signaling and an increase in metabolic activity (e.g. increase in glycolysis, oxidative phosphorylation (OXPHOS) and an increase in oxygen consumption rate/extracellular acidification rate (OCR/ECAR) ratio). Increased lipid rafts allow enhanced immune synapse formation for TCR signaling whilst enhanced metabolism allows efficient energy generation for T cell effector function. These results indicate that the mechanism(s) behind the increased T cell functionality following ACAT inhibition may be increased expansion of HBV-specific CD8+ T cells, lipid raft formation, TCR signaling and/or increase in metabolic activity.

### Example 2

### ACAT Inhibition Can Complement PD-1 Blockade

PD-1hi exhausted CD8+ T cells show reduced lipid rafts (see FIG. 3A). ACAT inhibition complements PD-1 blockade, i.e. ACAT inhibition alone or in combination with PD-1 blockade can be superior to PD-1 blockade alone (see FIG. 3B).

### Example 3

### ACAT Inhibition can Increase Functionality of T Follicular Helper Cells

T follicular helper cells (T_{FH}) are a subset of CD4+0 T cells (CD4+ CXCR5+PD-1+) required for antibody affinity maturation and to activate stimulate differentiation of B cells, and stimulate differentiation and antibody production to antibody-secreting cells. ACAT inhibition increases the frequency of T_{FH} (see FIG. 4A) and modulates the phenotype of T_{FH} by increasing expression of costimulatory molecules and activation markers (ICOS, OX40, 4-1BB, CD4OL; see FIG. 4B) aiding the ability of T_{FH} to activate and provide help to B cells.

### Example 4

### ACAT Inhibition Improves Ca²⁺ Mobilisation in Memory B Cells Isolated from Patients with CHB

Calcium mobilisation following BCR engagement can be used as a gauge of proximal B cell signaling following antigen engagement, required for differentiation and effector function in B cells. B cells in chronic HBV infection may have impaired BCR signaling upon antigen stimulation. ACAT inhibition improves Ca²⁺ mobilisation in classical memory B cells (CD27+CD21+) in patients with HBV (see FIG. 5A, B, C, E). ACAT inhibition does not affect Ca²⁺ mobilisation in classical memory B cells (CD27+CD21+) in healthy controls (see FIG. 5D). In CD19+ B cells, ACAT inhibition leads to a decrease of cholesteryl ester stored in neutral lipid that have been shown to have inhibitory effects in other immune cells, e.g. natural killer cells (see FIG. 5F).

### Example 5

### ACAT Inhibition Enhances the Functionality of TCR-Gene-Engineered T Cells

TCR-gene-engineered T cells are an immunotherapeutic approach for patients with HBV and HCC. ACAT inhibition enhances the cytokine production and lysis of tumour target cells by HBV-specific TCR-gene-engineered T cells (see FIG. 6).

### Example 6

### Direct Antiviral Effect of ACAT Inhibition

To determine the direct effects of ACAT inhibition, HepG2-NTCP cells were transduced with Ad-HBV at an MOI of 20 for 3 hrs. Inoculum was removed, cells washed 3 times with PBS and incubated with DMEM supplemented with the indicated doses of Avasimibe (AVS). Data is presented relative to untreated (see FIG. 7A).

In further experiments, DMSO-differentiated HepG2-NTCP cells were de-novo infected with HBV at MOI of 200. AVS was added at indicated doses every 3 days for a total of 6 days. Data is presented in FIG. 7C.

For both sets of experiments, extracellular HBV DNA in the supernatant was quantified by qPCR. ACAT inhibition demonstrates comparable antiviral effects in vitro as current best-in-class antiviral therapy—entecavir (see FIG. 7A+C).

### Example 7

### Capacity of ACAT Inhibition to Reduce HBV DNA and HBsAg

To determine the effects of ACAT inhibition immediately after infection, HepG2-NTCP cells were de-novo infected with HBV (as described previously). Inoculum was removed, cells washed 3 times with PBS and incubated with DMEM supplemented with the indicated doses of Avasimibe (AVS). Secreted HBsAg was quantitated by ELISA and the results are shown in FIG. 8A.

To determine the effects of ACAT inhibition when treatment is initiated after establishment of the infection, DMSO differentiated HepG2-NTCP-UB cells were infected with HBV at MOI of 200 and maintained for 6 days after which cells were treated with indicated doses of either Avasimibe or 1 μM ETV. Drugs were replenished every 3 days with treatment lasting for a total of 6 days. Extracellular HBV DNA was measured by qPCR and HBsAg quantified by ELISA and the results are shown in FIG. 8C and 8D.

### Example 8

### Combined Direct Antiviral and Immunomodulatory Effect of ACAT Inhibition

Current therapies in development for HBV either target the virus or the immune response, none can target both.

ACAT inhibition shows a combined antiviral and immunomodulatory effect when either the infected hepatocytes or the HBV-specific T cells are treated with ACAT inhibition (see FIG. 9). This leads to reduction of cccDNA and total intracellular DNA.

### Materials and Methods

### Lymphocyte Isolation

PBMC were isolated from heparinized blood by density centrifugation. Intrahepatic and intratumoural lymphocytes were isolated by mechanical disruption, incubation for 30 min at 37° C. in 0.01% collagenase IV and 0.001% DNAse I, followed by further mechanical disruption, removal of parenchymal cells by centrifugation on a 30% Percoll gradient and a density centrifugation.

Overnight- and Short-term Cell culture

To examine HBV- and HCC-specific CD8+ T cell responses from IHL and TIL, lymphocytes were stimulated with 1 μg/ml overlapping peptides (HBV: HBcAg, HBsAg, Pol; non-viral HCC: NY-ESO-1, AFP, MAGE-Al; HBV/HCC: both) for 18 h in the presence of 1 μg/ml Brefeldin A in cRPMI+10% FCS, followed by antibody staining and flow cytometric analysis. Where indicated, cells were treated with 0.1 μM of the ACAT inhibitor K-604.

To examine HBV-specific CD8+ and CD4+ T cell responses in the blood, PBMC where stimulated with 1 μg/ml of overlapping HBcAg peptides in cRPMI+10% FCS+20 U/ml IL-2. PBMC were expanded for 8 days with replenishment of IL-2 on day 2. PBMC were restimulated with 1 μg/ml peptide in the presence of 1 μg/ml Brefeldin A for 18 h on day 7, followed by antibody staining and flow cytometric analysis. Where indicated, cells were treated with 0.5 μM of the ACAT-inhibitor Avasimibe on d0, d2, d5, d7±2.5 μg/ml anti-PD-L1 and 2.5 μg/ml anti-PD-L2 on d0.

To examine T_{FH} responses, PBMC were cultured for 7d in cRPMI+10% FCS and cells were treated with 0.5 μM of the ACAT-inhibitor Avasimibe on d0, d2, d5, d7 where indicated.

### TCR-Gene-Engineered T Cells

TCR-gene-engineered CD8+ T cells specific for the HLA-A2-restricted C18 epitope of the HBcAg were stimulated with increasing doses of C18 peptide (0.0001 ng/ml to 0.05 ng/ml) presented by a HepG2 cell line (E:T ratio 1:1) for 18h in cRPMI+10% FCS in the presence of 1 μg/ml Brefeldin A, 100 U/ml IL-2, 10 ng/ml IL-7 and 10 ng/ml IL-15. 1 μM ACAT inhibitor Avasimibe was added where indicated. Antibody staining and flow cytometric analysis followed to determine cytokine production. Toxilight cytotoxicity assay was performed as described by the manufacturer to evaluate specific target cell lysis.

### Calcium Flux Assay

B cells were separated through magnetic separation and incubated with ACAT inhibitor Avasimibe for 18 h at 37° C. Cells were then stained for B cell memory phenotype and incubated with 1 μM Fluo-4 AM Dye at 37° C. for 30 min. Prior to analysis, cells were washed and rested in RPMI media supplemented with 250 mM probenecid for a minimum of 1 h. Cells were then analyzed via flow cytometry for 30 s to establish baseline prior to stimulation with F(ab′)₂-IgM/IgG/IgA (anti-BCR; 30 μg/ml), after which they were analyzed for a further 270s.

### Direct Antiviral Effect of Avasimibe

HepG2-NTCP cells were transduced with Ad-HBV (MOI 20) or de-novo infected with HBV (MOI 200) and incubated with the indicated doses of Avasimibe or Entecavir, which were replenished every 3 days. Drugs were applied for a total of 6 days either at the start of infection or after establishment of infection as indicated. Intracellular HBV DNA, cccDNA and extracellular HBV DNA, were quantified by qPCR, HBsAg was quantified by ELISA.

For the evaluation of a combined antiviral and immunomodulatory effect a co-culture experiment between HBV infected HepG2-NTCP cells and TCR-gene-engineered T cells was performed. Prior to the start of the co-culture, HepG2-NTCP were infected and treated with Avasimibe for 11days and TCR-gene-engineered T cells were treated with Avasimibe for 24 h. T-cells and Infected HepG2 cells were co-cultured for 72 h at an E:T ratio of 1:1 in the presence of Avasimibe where indicated. cccDNA and intracellular DNA were quantified by qPCR.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. An acyl-CoA: cholesterol acyltransferase (ACAT) inhibitor for use in the treatment of hepatitis B virus (HBV) infection in a subject.

2. An ACAT inhibitor for use in the treatment of HBV infection in a subject, wherein the infection is caused by HBV genotype C.

3. A therapeutic vaccine composition comprising an ACAT inhibitor for use in the treatment of HBV infection in a subject.

4. The ACAT inhibitor or therapeutic vaccine composition for use according to claim 1 or claim 3, wherein the infection is caused by HBV genotype A, HBV genotype B, HBV genotype C, HBV genotype D, HBV genotype E, HBV genotype F, HBV genotype G, HBV genotype H, HBV genotype I, and/or HBV genotype J.

5. The ACAT inhibitor or therapeutic vaccine composition for use according to any one of the preceding claims, wherein the HBV infection is chronic HBV infection (CHB).

6. The ACAT inhibitor or therapeutic vaccine composition for use according to any one of the preceding claims, wherein the subject is at risk of developing HBV-associated hepatocellular carcinoma (HCC), the subject has HBV-associated HCC or has previously had HBV-associated HCC.

7. The ACAT inhibitor or therapeutic vaccine composition for use according to any one of the preceding claims, wherein the ACAT inhibitor:
a) exhibits direct antiviral activity against HBV; and/or
b) enhances humoral immunity to HBV.

8. The ACAT inhibitor or therapeutic vaccine composition for use according to any one of the preceding claims, wherein the ACAT inhibitor:
a) exhibits direct antiviral activity against HBV;
b) enhances humoral immunity to HBV; and
c) enhances T cell immunity to HBV.

9. The ACAT inhibitor or therapeutic vaccine composition for use according to claim 7 or claim 8, wherein the ACAT inhibitor reduces HBV load and/or reduces HBV surface antigen (HBsAg) in a patient with HBV infection.

10. The ACAT inhibitor or therapeutic vaccine composition for use according to claim 7 or claim 8, wherein the ACAT inhibitor:
i) enhances the activity of B cells; and/or
ii) enhances the activity of CD4⁺ T cells, suitably CD4⁺ T_{FH} cells.

11. The ACAT inhibitor or therapeutic vaccine composition for use according to claim 7 or claim 8, wherein the ACAT inhibitor enhances the activity of HBV-specific CD8+ T cells.

12. The ACAT inhibitor or therapeutic vaccine composition for use according to any one of the preceding claims, wherein the ACAT inhibitor is selected from the group consisting of a small molecule inhibitor, a small inhibitory RNA (siRNA), a small hairpin RNA (shRNA), a micro RNA (miRNA), an antisense nucleic acid, an anti-ACAT antibody or fragment thereof, and combinations thereof.

13. The ACAT inhibitor for use according to any one of claims 1-12, wherein the ACAT inhibitor is avasimibe or K604.

14. The ACAT inhibitor or therapeutic vaccine composition for use according to any one of the preceding claims, wherein the ACAT inhibitor is administered to the subject in combination with at least one further pharmaceutically active agent.

15. The ACAT inhibitor for use according to claim 14, wherein the at least one further pharmaceutically active agent is selected from the group consisting of an antiviral, a therapeutic HBV vaccine, an immunostimulatory cytokine, a checkpoint inhibitor, TCR-gene-engineered T cells, activators of innate immunity, monoclonal or bispecific antibodies, CAR cells, soluble T cell receptors, or any combination thereof.

16. The therapeutic vaccine composition for use according to claim 14, wherein the at least one further pharmaceutically active agent is selected from the group consisting of an antiviral, an immunostimulatory cytokine, a checkpoint inhibitor, TCR-gene-engineered cells, activators of innate immunity, monoclonal or bispecific antibodies, CAR-T-cells, soluble T-cell receptors, or any combination thereof.

17. A therapeutic HBV vaccine composition comprising an ACAT inhibitor.

18. A TCR-gene-engineered cell or CAR cell having reduced ACAT activity and/or reduced ACAT expression for use in the treatment of HBV infection in a subject.

19. The TCR-gene-engineered cell or CAR cell for use according to claim 18, wherein the cell is engineered ex vivo to comprise a construct which inhibits ACAT expression or translation.

20. The TCR-gene-engineered cell or CAR cell for use according to claim 19 wherein the construct comprises a siRNA, a shRNA, a miRNA, an antisense nucleic acid against a nucleic acid encoding ACAT.

21. The TCR-gene-engineered cell or CAR cell for use according to claim 18-20, wherein the cell or subject is treated with an ACAT inhibitor selected from the group consisting of a small molecule inhibitor, a siRNA, a shRNA, a miRNA, an antisense nucleic acid, an anti-ACAT antibody or fragment thereof, and combinations thereof.

22. The TCR-gene-engineered cell or CAR cell for use according to any one of claims 18-21, wherein the infection is caused by HBV genotype A, HBV genotype B, HBV genotype C, HBV genotype D, HBV genotype E, HBV genotype F, HBV genotype G, HBV genotype H, HBV genotype I, and/or HBV genotype J.

23. The TCR-gene-engineered cell or CAR cell for use according to any one of claims 18-22, wherein the subject is at risk of developing HBV-associated hepatocellular carcinoma (HCC), the subject has HBV-associated HCC or has previously had HBV-associated HCC.

24. The TCR-gene-engineered cell or CAR cell for use according to any one of claims 18-23, wherein the cell is administered to the subject in combination with at least one further pharmaceutically active agent.

25. The TCR-gene-engineered cell or CAR cell for use according to claim 24, wherein the at least one further pharmaceutically active agent is selected from the group consisting of an an ACAT inhibitor, an antiviral, a therapeutic HBV vaccine, an immunostimulatory cytokine, a checkpoint inhibitor, activators of innate immunity, monoclonal or bispecific antibodies, soluble T cell receptors, or any combination thereof.


SEQUENCE LISTING (4 sequences)

SEQ ID NO: 1 - protein, 427 aa, Homo sapiens
MAVLAALLRSGARSRSPLLRRLVQEIRYVERSYVSKPTLKEVVIVSATRTPIGSFLGSLSLLPATKLGSIAIQGAIEKAGIPKEEVKEAYMGNVLQGGEGQAPTRQAVLGAGLPISTPCTTINKVCASGMKAIMMASQSLMCGHQDVMVAGGMESMSNVPYVMNRGSTPYGGVKLEDLIVKDGLTDVYNKIHMGSCAENTAKKLNIARNEQDAYAINSYTRSKAAWEAGKFGNEVIPVTVTVKGQPDVVVKEDEEYKRVDFSKVPKLKTVFQKENGTVTAANASTLNDGAAALVLMTADAAKRLNVTPLARIVAFADAAVEPIDFPIAPVYAASMVLKDVGLKKEDIAMWEVNEAFSLVVLANIKMLEIDPQKVNINGGAVSLGHPIGMSGARIVGHLTHALKQGEYGLASICNGGGGASAMLIQKL

SEQ ID NO: 2 - protein, 397 aa, Homo sapiens
MNAGSDPVVIVSAARTIIGSFNGALAAVPVQDLGSTVIKEVLKRATVAPEDVSEVIFGHVLAAGCGQNPVRQASVGAGIPYSVPAWSCQMICGSGLKAVCLAVQSIGIGDSSIVVAGGMENMSKAPHLAYLRTGVKIGEMPLTDSILCDGLTDAFHNCHMGITAENVAKKWQVSREDQDKVAVLSQNRTENAQKAGHFDKEIVPVLVSTRKGLIEVKTDEFPRHGSNIEAMSKLKPYFLTDGTGTVTPANASGINDGAAAVVLMKKSEADKRGLTPLARIVSWSQVGVEPSIMGIGPIPAIKQAVTKAGWSLEDVDIFEINEAFAAVSAAIVKELGLNPEKVNIEGGAIALGHPLGASGCRILVTLLHTLERMGRSRGVAALCIGGGMGIAMCVQRE

SEQ ID NO: 3 - DNA, 1537 bp, Homo sapiens
agtctacgcctgtggagccgatactcagccctctgcgaccatggctgtgctggcggcacttctgcgcagcggcgcccgcagccgcagccccctgctccggaggctggtgcaggaaataagatatgtggaacggagttatgtatcaaaacccactttgaaggaagtggtcatagtaagtgctacaagaacacccattggatcttttttaggcagcctttccttgctgccagccactaagcttggttccattgcaattcagggagccattgaaaaggcagggattccaaaagaagaagtgaaagaagcatacatgggtaatgttctacaaggaggtgaaggacaagctcctacaaggcaggcagtattgggtgcaggcttacctatttctactccatgtaccaccataaacaaagtttgtgcttcaggaatgaaagccatcatgatggcctctcaaagtcttatgtgtggacatcaggatgtgatggtggcaggtgggatggagagcatgtccaatgttccatatgtaatgaacagaggatcaacaccatatggtggggtaaagcttgaagatttgattgtaaaagacgggctaactgatgtctacaataaaattcatatgggcagctgtgctgagaatacagcaaagaagctgaatattgcacgaaatgaacaggacgcttatgctattaattcttataccagaagtaaagcagcatgggaagctgggaaatttggaaatgaagttattcctgtcacagttacagtaaaaggtcaaccagatgtagtggtgaaagaagatgaagaatataaacgtgttgattttagcaaagttccaaagctgaagacagttttccagaaagaaaatggcacagtaacagctgccaatgccagtacactgaatgatggagcagctgctctggttctcatgacggcagatgcagcgaagaggctcaatgttacaccactggcaagaatagtagcatttgctgacgctgctgtagaacctattgattttccaattgctcctgtatatgctgcatctatggttcttaaagatgtgggattgaaaaaagaagatattgcaatgtgggaagtaaatgaagcctttagtctggttgtactagcaaacattaaaatgttggagattgatccccaaaaagtgaatatcaatggaggagctgtttctctgggacatccaattgggatgtctggagccaggattgttggtcatttgactcatgccttgaagcaaggagaatacggtcttgccagtatttgcaatggaggaggaggtgcttctgccatgctaattcagaagctgtagacaacctctgctatttaaggagacaaccctatgtgaccagaaggcctgctgtaatcagtgtgactactgtgggtcagcttatattcagataagctgtttcattttttattattttctatgttaacttttaaaaatcaaaatgatgaaatcccaaaacattttgaaattaaaaataaatttcttcttctgcttttttcttggtaaccttgaaaa

SEQ ID NO: 4 - DNA, 1520 bp, Homo sapiens
agcttgcaggcagcgcagggcagacggcggcaggagaagcaagatgaatgcaggctcagatcctgtggtcatcgtctcggcggcgcggaccatcataggttccttcaatggtgccttagctgctgttcctgtccaggacctgggctccactgtcatcaaagaagtcttgaagagggccactgtggctccggaagatgtgtctgaggtcatctttggacatgtcttggcagcaggctgtgggcagaatcctgttagacaagccagtgtgggtgcaggaattccctactctgttccagcatggagctgccagatgatctgtgggtcaggcctaaaagctgtgtgccttgcagtccagtcaatagggataggagactccagcattgtggttgcaggaggcatggaaaatatgagcaaggctcctcacttggcttacttgagaacaggagtaaagataggtgagatgccactgactgacagtatactctgtgatggtcttacagatgcatttcacaactgtcatatgggtattacagctgaaaatgtagccaaaaaatggcaagtgagtagagaagatcaggacaaggttgcagttctgtcccagaacaggacagagaatgcacagaaagctggccattttgacaaagagattgtaccagttttggtgtcaactagaaaaggtcttattgaagttaaaacagatgagtttcctcgccatgggagcaacatagaagccatgtccaagctaaagccttactttcttactgatggaacgggaacagtcaccccagccaatgcttcaggaataaatgatggtgctgcagctgtcgttcttatgaagaagtcagaagctgataaacgtgggcttacacctttagcacggatagtttcctggtcccaagtgggtgtggagccttccattatgggaataggaccaattccagccataaagcaagctgttacaaaagcaggttggtcactggaagatgttgacatatttgaaatcaatgaagcctttgcagctgtctctgctgcaatagttaaagaacttggattaaacccagagaaggtcaatattgaaggaggggctatagccttgggccaccctcttggagcatctggctgtcgaattcttgtgaccctgttacacacactggagagaatgggcagaagtcgtggtgttgcagccctgtgcattgggggtgggatgggaatagcaatgtgtgttcagagagaatgaattgcttaaactttgaacaacctcaatttctttttaaactaataaagtactaggttgcaatatgtgaaatcagaggaccaaagtacagatggaaaccatttcctacatcacaaaaacccaagtttacagcttgtactttactttaatgtgtaatactcaactcaaggtacaagacaattgcatttaacattgttataaataaaaggaacatcagatcaatcattaagggctccagagtgaacagcatcttcataacttccatgtttatcatctttactttctg